SEQUENCE LISTING (3 sequences)

SEQ ID NO: 1 - protein, 9 aa, Synthetic construct
EDDMMEVPY

SEQ ID NO: 2 - protein, 3571 aa, Homo sapiens
MWPRLAFCCWGLALVSGWATFQQMSPSRNFSFRLFPETAPGAPGSIPAPPAPGDEAAGSRVERLGQAFRRRVRLLRELSERLELVFLVDDSSSVGEVNFRSELMFVRKLLSDFPVVPTATRVAIVTFSSKNYVVPRVDYISTRRARQHKCALLLQEIPAISYRGGGTYTKGAFQQAAQILLHARENSTKVVFLITDGYSNGGDPRPIAASLRDSGVEIFTFGIWQGNIRELNDMASTPKEEHCYLLHSFEEFEALARRALHEDLPSGSFIQDDMVHCSYLCDEGKDCCDRMGSCKCGTHTGHFECICEKGYYGKGLQYECTACPSGTYKPEGSPGGISSCIPCPDENHTSPPGSTSPEDCVCREGYRASGQTCELVHCPALKPPENGYFIQNTCNNHFNAACGVRCHPGFDLVGSSIILCLPNGLWSGSESYCRVRTCPHLRQPKHGHISCSTREMLYKTTCLVACDEGYRLEGSDKLTCQGNSQWDGPEPRCVERHCSTFQMPKDVIISPHNCGKQPAKFGTICYVSCRQGFILSGVKEMLRCTTSGKWNVGVQAAVCKDVEAPQINCPKDIEAKTLEQQDSANVTWQIPTAKDNSGEKVSVHVHPAFTPPYLFPIGDVAIVYTATDLSGNQASCIFHIKVIDAEPPVIDWCRSPPPVQVSEKVHAASWDEPQFSDNSGAELVITRSHTQGDLFPQGETIVQYTATDPSGNNRTCDIHIVIKGSPCEIPFTPVNGDFICTPDNTGVNCTLTCLEGYDFTEGSTDKYYCAYEDGVWKPTYTTEWPDCAKKRFANHGFKSFEMFYKAARCDDTDLMKKFSEAFETTLGKMVPSFCSDAEDIDCRLEENLTKKYCLEYNYDYENGFAIGPGGWGAANRLDYSYDDFLDTVQETATSIGNAKSSRIKRSAPLSDYKIKLIFNITASVPLPDERNDTLEWENQQRLLQTLETITNKLKRTLNKDPMYSFQLASEILIADSNSLETKKASPFCRPGSVLRGRMCVNCPLGTYYNLEHFTCESCRIGSYQDEEGQLECKLCPSGMYTEYIHSRNISDCKAQCKQGTYSYSGLETCESCPLGTYQPKFGSRSCLSCPENTSTVKRGAVNISACGVPCPEGKFSRSGLMPCHPCPRDYYQPNAGKAFCLACPFYGTTPFAGSRSITECSSFSSTFSAAEESVVPPASLGHIKKRHEISSQVFHECFFNPCHNSGTCQQLGRGYVCLCPLGYTGLKCETDIDECSPLPCLNNGVCKDLVGEFICECPSGYTGQRCEENINECSSSPCLNKGICVDGVAGYRCTCVKGFVGLHCETEVNECQSNPCLNNAVCEDQVGGFLCKCPPGFLGTRCGKNVDECLSQPCKNGATCKDGANSFRCLCAAGFTGSHCELNINECQSNPCRNQATCVDELNSYSCKCQPGFSGKRCETEQSTGFNLDFEVSGIYGYVMLDGMLPSLHALTCTFWMKSSDDMNYGTPISYAVDNGSDNTLLLTDYNGWVLYVNGREKITNCPSVNDGRWHHIAITWTSANGIWKVYIDGKLSDGGAGLSVGLPIPGGGALVLGQEQDKKGEGFSPAESFVGSISQLNLWDYVLSPQQVKSLATSCPEELSKGNVLAWPDFLSGIVGKVKIDSKSIFCSDCPRLGGSVPHLRTASEDLKPGSKVNLFCDPGFQLVGNPVQYCLNQGQWTQPLPHCERISCGVPPPLENGFHSADDFYAGSTVTYQCNNGYYLLGDSRMFCTDNGSWNGVSPSCLDVDECAVGSDCSEHASCLNVDGSYICSCVPPYTGDGKNCAEPIKCKAPGNPENGHSSGEIYTVGAEVTFSCQEGYQLMGVTKITCLESGEWNHLIPYCKAVSCGKPAIPENGCIEELAFTFGSKVTYRCNKGYTLAGDKESSCLANSSWSHSPPVCEPVKCSSPENINNGKYILSGLTYLSTASYSCDTGYSLQGPSIIECTASGIWDRAPPACHLVFCGEPPAIKDAVITGNNFTFRNTVTYTCKEGYTLAGLDTIECLADGKWSRSDQQCLAVSCDEPPIVDHASPETAHRLFGDIAFYYCSDGYSLADNSQLLCNAQGKWVPPEGQDMPRCIAHFCEKPPSVSYSILESVSKAKFAAGSVVSFKCMEGFVLNTSAKIECMRGGQWNPSPMSIQCIPVRCGEPPSIMNGYASGSNYSFGAMVAYSCNKGFYIKGEKKSTCEATGQWSSPIPTCHPVSCGEPPKVENGFLEHTTGRIFESEVRYQCNPGYKSVGSPVFVCQANRHWHSESPLMCVPLDCGKPPPIQNGFMKGENFEVGSKVQFFCNEGYELVGDSSWTCQKSGKWNKKSNPKCMPAKCPEPPLLENQLVLKELTTEVGVVTFSCKEGHVLQGPSVLKCLPSQQWNDSFPVCKIVLCTPPPLISFGVPIPSSALHFGSTVKYSCVGGFFLRGNSTTLCQPDGTWSSPLPECVPVECPQPEEIPNGIIDVQGLAYLSTALYTCKPGFELVGNTTTLCGENGHWLGGKPTCKAIECLKPKEILNGKFSYTDLHYGQTVTYSCNRGFRLEGPSALTCLETGDWDVDAPSCNAIHCDSPQPIENGFVEGADYSYGAIIIYSCFPGFQVAGHAMQTCEESGWSSSIPTCMPIDCGLPPHIDFGDCTKLKDDQGYFEQEDDMMEVPYVTPHPPYHLGAVAKTWENTKESPATHSSNFLYGTMVSYTCNPGYELLGNPVLICQEDGTWNGSAPSCISIECDLPTAPENGFLRFTETSMGSAVQYSCKPGHILAGSDLRLCLENRKWSGASPRCEAISCKKPNPVMNGSIKGSNYTYLSTLYYECDPGYVLNGTERRTCQDDKNWDEDEPICIPVDCSSPPVSANGQVRGDEYTFQKEIEYTCNEGFLLEGARSRVCLANGSWSGATPDCVPVRCATPPQLANGVTEGLDYGFMKEVTFHCHEGYILHGAPKLTCQSDGNWDAEIPLCKPVNCGPPEDLAHGFPNGFSFIHGGHIQYQCFPGYKLHGNSSRRCLSNGSWSGSSPSCLPCRCSTPVIEYGTVNGTDFDCGKAARIQCFKGFKLLGLSEITCEADGQWSSGFPHCEHTSCGSLPMIPNAFISETSSWKENVITYSCRSGYVIQGSSDLICTEKGVWSQPYPVCEPLSCGSPPSVANAVATGEAHTYESEVKLRCLEGYTMDTDTDTFTCQKDGRWFPERISCSPKKCPLPENITHILVHGDDFSVNRQVSVSCAEGYTFEGVNISVCQLDGTWEPPFSDESCSPVSCGKPESPEHGFVVGSKYTFESTIIYQCEPGYELEGNRERVCQENRQWSGGVAICKETRCETPLEFLNGKADIENRTTGPNVVYSCNRGYSLEGPSEAHCTENGTWSHPVPLCKPNPCPVPFVIPENALLSEKEFYVDQNVSIKCREGFLLQGHGIITCNPDETWTQTSAKCEKISCGPPAHVENAIARGVHYQYGDMITYSCYSGYMLEGFLRSVCLENGTWTSPPICRAVCRFPCQNGGICQRPNACSCPEGWMGRLCEEPICILPCLNGGRCVAPYQCDCPPGWTGSRCHTAVCQSPCLNGGKCVRPNRCHCLSSWTGHNCSRKRRTGF

SEQ ID NO: 3 - protein, 21 aa, Homo sapiens
QGYFEQEDDMMEVPYVTPHPP

(19) United States
(12) Patent Application Publication
(10) Pub. No.: US 2026/0077011 A1
(43) Pub. Date: Mar. 19, 2026

(54) INTEGRIN INHIBITORS AS THERAPY FOR OBESITY DISORDERS

(71) Applicant: RAMOT AT TEL-AVIV UNIVERSITY LTD., Tel Aviv (IL)

(72) Inventors: Dafna BENAYAHU, Tel Aviv (IL); Nadav KISLEV, Tel Aviv (IL)

(21) Appl. No.: 19/108,282

(22) PCT Filed: Sep. 4, 2023
(86) PCT No.: PCT/IL2023/050943

§ 371 Date: Mar. 3, 2025

Related U.S. Application Data
(60) Provisional application No. 63/403,766, filed on Sep. 4, 2022.
(60) Provisional application No. 63/437,794, filed on Jan. 9, 2023.

(51) Int. Cl. A61K 38/04; A61P 3/04; A61P 3/10; A61P 19/08; A61P 29/00

(52) U.S. Cl. CPC A61K 38/04; A61P 3/04; A61P 3/10; A61P 19/08; A61P 29/00

(57) ABSTRACT

Methods for inhibiting or preventing activation of a macrophage, by a receptor antagonist of α9β1 integrin are provided. Further provided are methods for treating or preventing an inflammatory disease or a metabolic disorder, including a metabolic syndrome, in a subject in need thereof, and a pharmaceutical composition comprising a peptide comprising the amino acid sequence: EDDMMEVPY, for use in treatment or prevention of an inflammatory disease or a disorder, in a subject in need thereof.

### CROSS-REFERENCE TO RELATED APPLICATIONS

This application claims the benefit of priority of U.S. Provisional Patent Application No. 63/403,766, filed Sep. 4, 2022, and of U.S. Provisional Patent Application No. 63/437,794 filed Jan. 9, 2023, both titled “INTEGRIN INHIBITORS AS THERAPY FOR OBESITY DISORDERS”. The contents of both applications are hereby incorporated by reference in their entirety.

### REFERENCE TO AN ELECTRONIC SEQUENCE LISTING

The contents of the electronic sequence listing (RMT-P-024-PCT ST26.xml; size: 7,189 bytes; and date of creation: Aug. 10, 2023) is herein incorporated by reference in its entirety.

### FIELD OF INVENTION

The present invention is in the field of obesity and metabolic diseases.

### BACKGROUND

Obesity is a metabolic disorder that is increasing globally and has been linked to major causes of death, such as diabetes and heart disease. Adipose tissue dysfunction lies at the center of obesity pathogenesis. It is associated with surpassing the storage capacity of adipose cells, which leads to metabolic impairment and systemic meta-inflammation. Proteins involved in this process have drawn much attention due to their ability to affect the inflammatory response and alter the metabolic state. Sushi, von Willebrand factor type A, EGF, and pentraxin 1 (SVEP1) is a 400 kDa multidomain protein with potential metabolic and immune functions. SVEP1 is an adhesion molecule found mainly in the extracellular matrix of mesenchymal tissues and was previously shown to play a role in several metabolic conditions such as diabetes, obesity, and cardiovascular diseases. In vivo mice model revealed that heterozygous KO of SVEP1 results in decreased fat mass and body mass. Moreover, the expression of SVEP1 gene was correlated with an increased prevalence of diabetes, hypertension, and coronary artery disease.

SVEP1 main studied receptor is integrin α9β1, an integrin molecule that also plays a role in adhesion and migration. The previously studied binding site of SVEP1 to the integrin is a nine amino acids long peptide with potential inhibitory capabilities over the integrin's axes. This 9 amino acid peptide composition and methods of usage, in part for inducing stem cells in antiproliferative or undifferentiated state, was previously disclosed. Previous single-cell data from human adipose tissue demonstrate that integrin α9 (ITAG) is expressed by endothelial cells, mast cells and macrophages. A mutation in alpha 4 integrin has been previously demonstrated to affect monocytes and monocytes—derived macrophages in a murine model of high fat diet (HFD). Nonetheless, there is no knowledge about the expression of α9β1 integrin receptor in different macrophage subpopulations of the adipose tissue, and specifically, in these subpopulations within a dysfunctional or an inflamed adipose tissue. There is an unmet need for new methods for treating adipose tissue dysfunction, and the consequentially obesity related diseases, comprising metabolic diseases.

### SUMMARY

According to one aspect, there is provided a method for inhibiting or preventing activation of a macrophage, the method comprising contacting the macrophage with a peptide of 9 to 15 amino acids comprising the amino acid sequence: EDDMMEVPY (SEQ ID NO: 1), thereby inhibiting or preventing activation of the macrophage.

According to another aspect, there is provided a method for treating or preventing an inflammatory disease or disorder, in a subject in need thereof, the method comprising administering to the subject a therapeutically effective amount of a pharmaceutical composition comprising a peptide of 9 to 15 amino acids comprising the amino acid sequence: EDDMMEVPY (SEQ ID NO: 1), thereby treating or preventing inflammatory disease or disorder, in the subject.

According to another aspect, there is provided a pharmaceutical composition comprising a peptide of 9 to 15 amino acids comprising the amino acid sequence: EDDMMEVPY (SEQ ID NO: 1), for use in treatment or prevention of an inflammatory disease or a disorder, in a subject in need thereof.

According to another aspect there is provided a method for treating or preventing a metabolic disease or disorder, in a subject in need thereof, the method comprising administering to the subject a therapeutically effective amount of a pharmaceutical composition comprising a peptide of 9 to 15 amino acids comprising the amino acid sequence: EDDMMEVPY (SEQ ID NO: 1), thereby treating or preventing a metabolic disease or disorder, in the subject.

In some embodiments, the macrophage is characterized by expression of integrin α9β1.

In some embodiments, the inhibiting or preventing activation comprises reducing at least one parameter selected from the group consisting of: adhesion, migration, cell signaling, expression and/or secretion of at least one pro-inflammatory cytokine, and any combination thereof, of the macrophage.

In some embodiments, the macrophage is a macrophage of a subject.

In some embodiments, the macrophage comprises a descendant cell of a macrophage.

In some embodiments, the descendant cell of a macrophage comprises a giant cell.

In some embodiments, the descendant cell of a macrophage comprises an osteoclast.

In some embodiments, the contacting comprises administering to the subject a therapeutically effective amount of the peptide.

In some embodiments, the macrophage is an adipose tissue macrophage (ATM).

In some embodiments, the macrophage is an M1 or M1-like macrophage.

In some embodiments, the subject is afflicted with an inflammatory disease or disorder.

In some embodiments, the inflammatory disease or disorder comprises a metabolic syndrome.

In some embodiments, the metabolic syndrome comprises any one of: obesity, pre-diabetes, diabetes, hyperglycemia, diabetic dyslipidemia, hyperlipidemia, hypertriglyceridemia, hyper-fattyacidemia, hypercholesterolemia, hyperinsulinemia, insulin-resistance, hypertension, bone resorption, and any combination thereof.

In some embodiments, the subject is characterized by having a macrophage being: (i) characterized by expression of integrin α9β1; (ii) an M1 or M1-like macrophage, or (iii) a combination of (i) and (ii).

In some embodiments, the treating or preventing comprises reducing at least one parameter selected from the group consisting of: adhesion, migration, cell signaling, expression and/or secretion of at least one pro-inflammatory cytokine, and any combination thereof, of a macrophage in the subject.

In some embodiments, the administering comprises: systemically administering, intravenously administering, oral administering, transdermal administering, or any combination thereof.

In some embodiments, the peptide is an antagonist of integrin α9β1.

In some embodiments, the peptide is incapable of inducing or promoting signaling via integrin α9β1.

In some embodiments, a level of integrin α9β1 signaling being induced by the peptide is about 0.0001%-1% the level of integrin α9β1 signaling being induced by a control ligand of integrin α9β1.

In some embodiments, the control ligand of integrin α9β1 comprises an amino acid sequence set forth in SEQ ID NO: 2.

In some embodiments, the metabolic disease or disorder comprises any one of: obesity, pre-diabetes, diabetes, hyperglycemia, diabetic dyslipidemia, hyperlipidemia, hypertriglyceridemia, hyper-fattyacidemia, hypercholesterolemia, hyperinsulinemia, insulin-resistance, hypertension, or any combination thereof.

In some embodiments, treating or preventing comprises at least one parameter selected from: reducing weight, reducing adipose tissue volume or weight, increasing insulin sensitivity, reducing glucose level, reducing the number of inflammatory cells, reducing the number of ATMs, or any combination thereof, in the subject.

In some embodiments, reducing weight comprises increasing rate of weight loss.

In some embodiments, administering comprises injecting into a fatty tissue of the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1F include graphs, micrographs, and fluorescent micrographs showing Sushi, von Willebrand factor type A, EGF, and pentraxin 1 (SVEP1) and Integrin α9β1 expression patterns in adipose tissue. (1A) The GTeX expression profile of SVEP1 in human connective tissues samples, the data was normalized with the RMSE tissue aware method. (1B) Representative image of SVEP1 and DAPI in Visceral adipose tissue VAT, the dotted line square represents the magnified pictures (scale bar, 116 μm). (1C) Quantitative PCR (qPCR) analysis of SVEP1 mRNA levels in high-fat diet-fed (HFD) mice compared to chow-fed mice (CHD, n=5), and fluorescent intensity analysis for SVEP1 in high-fat diet-fed (HFD) mice compared to chow-fed mice (n=4). (1D) Representative image of ITGA9, SVEP1, and DAPI in VAT the dotted line square represents the magnified pictures (scale bar, 116 μm). (1E) Expression profiles in human single cell RNA sequencing (scRNAseq) adipose tissue experiment that was conducted by Emont et al., 2022, right panels show SVEP1 and ITGA9 in the different clusters. (1F) ITGA9, and SVEP1 expression levels in human adipose tissue and expression plots of SVEP1 (adipocytes) and ITGA9 (macrophages) in scRNAseq of different BMI ranges, significance was calculated using a two-tailed unpaired student's t-test, error bars represent mean±SEM.

FIGS. 2A-2G include graphs, micrographs, and fluorescent micrographs showing characterization of ITGA9 in adipose tissue macrophages. (2A) Body weights of control (CHD) mice and high-fat diet (HFD) fed mice (n=10-12 per group) after 0, 6 and 12 weeks of feeding. Stromal vascular fractions from epidydimal adipose tissues of HFD and CHD mice (n=3 in each group) were isolated and analyzed by flow cytometry. (2B) Quantification of CD45 positive cells (n=3). (2C) Flow cytometry dot plot of F4/80 and CD11b profiles in CD45-positive cells (2D) Quantification of F4/80^{high} CD11b+ and F4/80^{int} CD11b+ subpopulations (n=3). (2E) Flow cytometry dot plots and percentage quantification of the adipose tissue macrophage subpopulations in HFD and CHD mice. The upper panel shows the MHCII and CD11c profiles in F4/80^{high} macrophages, and the lower panel shows the Ly6C and MHCII profiles of F4/80^{int} cells. (2F) Representative image of ITGA9, and F4/80 in Visceral adipose tissue (VAT), scale bar, 116 μm. (2G) Quantification of ITGA9 mean fluorescence intensity of adipose tissue macrophages subpopulations in lean (CHD) or obese (HFD) VAT measured by flow cytometry (n=3), significance was calculated using a two-tailed unpaired student's t-test, error bars represent mean±SEM.

FIGS. 3A-3E include graphs and plots showing trajectory analysis of the adipose tissue macrophages subpopulations. (3A) Tree-shaped trajectory analysis of flow cytometry data of adipose tissue macrophages subpopulations in VAT of 16-week-old mice, the different subtypes and clusters were constructed by a Uniform Manifold Approximation and Projection for Dimension Reduction (UMAP). In the tree plot with the marked yellow annotation, the color of the branches represents the different clusters (right panel) and expression markers levels in the tree plot (left panel). (3B) UMAP dot plot visualization of the cells in HFD and CHD samples, and (3C) UMAP expression dot plots of the different markers. (3D) General normalized ITGA9 expression in the HFD and CHD samples (n=3 samples in each group). (3E) UMAP expression dot plot of ITGA9 in HFD and CHD and normalized ITGA9 expression in the different subpopulations in the HFD and CHD samples (n=3 samples in each group).

FIGS. 4A-4D include structural illustrations showing that protein-peptide interaction analysis of integrin α9β1 and its ligands suggest a specific binding site. (4A) Alignment of Integrin α9β1 alphafold predication with a PDB structure of integrin α5β1 (3VI4). (4B) A Scannet analysis of potential structure-based protein binding site prediction of integrin α9β1, and a protein-protein interaction interface analysis. (4C) A predicted model of SVEP1-based peptide interaction with integrin α9β1, with a close-up of interacting a.a. (4D) An overlay of various binding site-based peptides of five ligands of integrin (Tenascin-C, Emilin-1, SVEP1, Osteopontin, and VCAM-1), with a close up of the binding domain.

FIGS. 5A-5M include fluorescent micrographs and graphs showing characterization of the peptide's binding properties. (5A) Representative immunofluorescent confocal images of Raw264.7 cells stained for F4/80 and ITGA9, the dotted line square represents the magnified pictures, scale bar 116 μm. (5B) Flow cytometry quantification of LPS-activated RAW264.7 cells, ITGA9 MFI levels in LPS-activated RAW264.7 cells (4 hours), significance was calculated by unpaired t-test analysis. (5C) Calculated dose-response curve of Peptide-FITC positive RAW264.7 cells, measured in flow cytometry and time curve of the averaged mean fluorescent intensity (MFI) of RAW264.7 cells incubated with the labeled peptide, the MFI was calculated by flow cytometry. (5D) Representative images of RAW264.7 cells incubated with FITC-peptide (30 μM) for 15 and 60 minutes with and without an unlabeled peptide, scale bar=100 μm, and quantification of fluorescence intensity of RAW264.7 cells incubated with FITC-peptide (30 μM) for 15 and 60 minutes, w/o the unlabeled peptide, significance was calculated by using an ordinary one-way ANOVA. (5E) Representative images of Scrambled (siSCR) and ITGA9 KD (siITGA9) RAW264.7 cells incubated with FITC-peptide (30 μM) for 60 minutes. (5F) ITGA9 fluorescence intensity histogram in Scrambled and ITGA9 KD (Red) Raw264.7 cells incubated with FITC-peptide (30 μM) for 60 minutes, measured by flow cytometry. (5G) FITC-Peptide mean fluorescence intensity quantification in Scrambled and ITGA9 KD Raw 264.7 cells incubated with FITC-peptide (30 μM) for 60 minutes, measured by flow cytometry, and quantification of the ITGA9+FITC-Peptide percentage of cells. (5H) Adhesion rate of cells exposed to different concentrations (7.5 μM and 30 μM) of the peptide normalized to DMSO-exposed cells. Significance was calculated using an ordinary one-way ANOVA (5I) mean speed of cells that were treated with the peptide and untreated cells (n=40), significance was calculated using a two-tailed unpaired student's t-test. (5J) Western blot analysis of phosphorylated and total ERK in untreated and treated cells after 30 minutes. (5K) Experimental design; cells were untreated (CTL) and treated with the Peptide, LPS, LPS, and peptide. (5L) Griess assay analysis of nitrite concentration in the medium after 8 hours (n=3), significance was calculated using an ordinary one-way ANOVA. (5M) qPCR analysis of representative pro-inflammatory genes, significance was calculated using an ordinary one-way ANOVA, error bars represent means±SEM.

FIGS. 6A-6B include vertical bar graphs showing that the peptide does not affect the metabolic activity and cell death rate in RAW264.7 cells. (6A) Metabolic activity analysis of RAW264.7 cells using XTT after 3, 8, and 24 hours of peptide (1, 10, 30, 120 μM) exposure (n=3). (6B) Viability of RAW264.7 cells after 8, and 24 hr of peptide and DMSO exposure was determined using PI staining followed by flow cytometry analysis (n=4). Significance was calculated using an ordinary one-way ANOVA, error bars represent means±SEM.

FIGS. 7A-7D include vertical bar graphs and a photograph showing that the peptide effectively inhibits adhesion and inflammation activation in bone marrow derived macrophages. (7A) Adhesion rate of cells exposed to different concentrations (1, 10, 30, 60 and 120 μM) of the peptide normalized to untreated cells. Significance was calculated using an ordinary one-way ANOVA (n=4). (7B) Griess assay analysis of nitrite concentration in the medium after 8 hours (n=8), significance was calculated using an ordinary one-way ANOVA. (7C) Western blot analysis of phosphorylated and total ERK in untreated and treated cells after 30 minutes, and the percentage of phosphorylation quantification (n=4), significance was calculated using an unpaired-student's t-test. (7D) qPCR analysis of representative pro-inflammatory genes after eight hours of LPS (100 nM) with or w/o the peptide (30 μM) compared to untreated cells (n=3-4), significance was calculated using an ordinary one-way ANOVA, error bars represent means±SEM.

FIGS. 8A-8H includes graphs showing that SVEP1, an extracellular matrix protein highly expressed in adipose tissue, affects glucose metabolism in adipose tissue. (8A) SVEP1 mRNA levels in inguinal and epididymal adipose tissues, liver, and muscle of 14 weeks-old wild type (SVEP1^{+/+}) and heterozygous KO (SVEP1^{+/-}) mice (n=4-6). (8B) Body weights of 14 weeks-old wild type (SVEP1) and heterozygous KO (SVEP1^{+/-}) mice. (8C) Relative organ/body ratios in inguinal and epididymal adipose tissues, liver, and muscle of 14 weeks-old wild type (SVEP1^{+/+}) and heterozygous KO (SVEP1^{+/-}) mice (n=4-6). (8D) Inguinal to epidydimal adipose tissue ratio in wild type (SVEP1^{+/+}) and heterozygous KO (SVEP1^{+/-}) mice (n=4-6). (8E) glucose levels at fasting (18 hours fast) and fed state. (8F-8G) Intraperitoneal glucose (8F) and insulin tolerance tests (8G) on wild type (SVEP1^{+/+}) and heterozygous KO (SVEP1^{+/-}) mice at 14 weeks of age. Significance was calculated using two-way ANOVA. Area-under-the-curve (AUC) measurements are shown (n=3-6 mice per group). (8H) qPCR analysis of pro inflammatory genes from epidydimal adipose tissue of WT and KO mice (n=4). Unless stated otherwise, significance was calculated using a two-tailed unpaired student's t-test; error bars represent mean±SEM.

FIGS. 9A-9E include schemes, graphs, and a fluorescent micrograph showing the peptide in an ex vivo adipose tissue. (9A) A non-limiting scheme of an experimental design; Epidydimal adipose tissues were isolated from HFD-fed mice and then incubated in a trans-well culturing system with the peptide. (9B) qPCR analysis of representative pro-inflammatory genes, significance was calculated using a two-tailed unpaired student's t-test. (9C) Representative immunofluorescent confocal images of ex vivo VAT incubated with the FITC-peptide for 1 hour, scale bar=26 μm. (9D) A non-limiting scheme of an experimental design of the intracellular cytokine assay; the isolated tissues were incubated with LPS or LPS+Peptide for 16 hours, and in the last 4 hours of the incubation, brefeldin A was supplemented. The SVFs were then isolated from the tissues, fixated, and stained for extracellular and intracellular markers subsequentially. (9E) Quantification of positive cell for TNFα, IL6, and both, obtained by a flow cytometry dot plots of TNFα versus IL6 profiles of immune cells (CD45+; Left) or adipose tissue macrophages in tissues (F4/80+CD11b+; Right) incubated with LPS+Peptide or LPS. Unless stated otherwise, significance was calculated using a two-tailed unpaired student's t-test; error bars represent mean±SEM.

FIGS. 10A-10K include a scheme, graphs, and fluorescent micrographs showing that the peptide improves systemic glucose homeostasis, weight, and adipose tissue inflammation in mice. (10A) A non-limiting scheme of an experimental design; 6 weeks old c57bl/6 mice were fed an HFD for 12 weeks. The mice were injected with 200 μl of the peptide or vehicle into both sides of their epidydimal fat pads at 24 hours intervals in the last 72 hours of the experiment. (10B) Body weight of peptide-injected mice and control mice before the peptide injection and 24/48 hours after the first injection (n=6). (10C) Body weight differences of peptide-injected mice and control mice before the peptide injection and at the end of the experiment (n=6). (10D) Weights of the epidydimal fat pads, livers, and brown adipose tissues (n=6). (10E) Fasting glucose levels (n=6). (10F) Glucose tolerance tests on peptide-injected and control groups and area under the curve calculation (n=6). (10G) Flow cytometry dot plots and percentage quantification graphs of CD11b and F4/80 in CD45+ cells of the vehicle and peptide-treated groups (n=5). (10H) Representative image of CD206 (Red), F4/80 (magenta), and DAPI (blue) in vehicle and peptide-treated visceral adipose tissue (VAT) (scale bar, 310 μm) and quantification of the number of crown-like structures (macrophages aggregates) per field, and the percentage of CD206 positive cells out of F4/80+ cells (f=8, n=3). (10I) Western blot analysis of phosphorylated and total ERK in vehicle and peptide-treated visceral adipose tissue (VAT) and liver, and the percentage of phosphorylation quantification in VAT (n=3). (10J) qPCR analysis of adipogenic and proinflammatory genes from epidydimal adipose tissue of vehicle and peptide injected mice (n=6). (10K) qPCR analysis of liver markers and proinflammatory genes from livers of vehicle and peptide injected mice (n=4). Unless stated otherwise, significance was calculated using a two-tailed unpaired student's t-test; error bars represent mean±SEM.

FIGS. 11A-11B include a scheme, micrographs, and vertical bar graphs. (11A) A non-limiting schematic illustration of giant cells and osteoclast formation. (11B) RAW264.7 cells (left) or when cultured with Rank-L are TRAP+ (right). In presence of peptide (left lower panel), the peptide reduced the formation of giant cells, was quantification after 4 days is provided in the graph. The group co-incubated with Rank-L and the peptide displayed significantly lower levels of osteoclast cells formation, and the cells remain in their progenitor state. Significance was calculated using an ordinary one-way ANOVA, error bars represent means±SEM. The same effect was achieved on BMDM cultured cells.

### DETAILED DESCRIPTION

The present invention, in some embodiments, is partially based on the surprising finding, that a tissue specific macrophage subpopulation, comprising adipose tissue macrophages (ATMs), is the primarily subpopulation that expresses integrin α9 (ITAG9) or the complex integrin α9β1 receptor, among adipose tissue myeloid cells. In some embodiments, the inventors found that stimulated ATMs, that are known to be abundantly present in a dysfunctional or inflamed adipose tissue, express increased levels of the α9β1 integrin. In some embodiments, it was found that the SVEP1-based inhibitory peptide comprising the 9 amino acid: EDDMMEVPY (SEQ ID NO: 1), specifically binds to α9β1 integrin receptor. In some embodiments, it was surprisingly found that the inhibitory peptide possesses the ability to inhibit ATMs activation, comprising adhesion, migration, an activated signaling pathway, or expression and/or secretion of at least one pro-inflammatory cytokine. The present invention, in some embodiments, is further based, at least in part, on the findings that the peptide was found to bind an adipose tissue ex vivo and suppress an inflammatory response in an ex-vivo system. Injection of the peptide to high fed diet (HFD) mice, was shown to lead to reduction of weight, reduced epididymal fat, improved insulin resistance, and alleviated metabolic burden. In some embodiments, inhibited activation of adipose tissue macrophage (ATMs) by the inhibitory peptide could be used for treating pathologic inflammatory conditions, in a subject in need thereof, comprising metabolic condition or disease.

The present invention, in some embodiments, provides a method for inhibiting or preventing activation of a myeloid cell by contacting the cell with a peptide comprising the amino acid sequence: EDDMMEVPY (SEQ ID NO: 1).

In some embodiments, the peptide consists of SEQ ID NO: 1.

In some embodiments, the peptide disclosed herein binds to the receptor integrin α9β1. In some embodiments, the peptide comprises or consists of 9 to 15 amino acids. In some embodiments, the peptide comprises the amino acids set forth in SEQ ID NO: 1, and optionally further comprises additional 1-2, 1-3, 1-4, 1-5, 1-6, 2-3, 2-4, 2-5, 2-6, 3-4, 3-5, 3-6, 4-5, 4-6, or 5-6, amino acids. In some embodiments, the additional amino acids are from the polypeptide: Sushi, von Willebrand factor type A, EGF and pentraxin domain-containing protein 1 (SVEP1). In some embodiments, SVEP1 comprises a mammalian SVEP1. In some embodiments, SVEP1 comprises a human SVEP1 (UniProtKB #Q4LDE5).

In some embodiments, human SVEP1 comprises the amino acid sequence:

(SEQ ID NO: 2)
MWPRLAFCCWGLALVSGWATFQQMSPSRNFSFRLFPETAPGAPGSIPAP
PAPGDEAAGSRVERLGQAFRRRVRLLRELSERLELVFLVDDSSSVGEVN
FRSELMFVRKLLSDFPVVPTATRVAIVTFSSKNYVVPRVDYISTRRARQ
HKCALLLQEIPAISYRGGGTYTKGAFQQAAQILLHARENSTKVVFLITD
GYSNGGDPRPIAASLRDSGVEIFTFGIWQGNIRELNDMASTPKEEHCYL
LHSFEEFEALARRALHEDLPSGSFIQDDMVHCSYLCDEGKDCCDRMGSC
KCGTHTGHFECICEKGYYGKGLQYECTACPSGTYKPEGSPGGISSCIPC
PDENHTSPPGSTSPEDCVCREGYRASGQTCELVHCPALKPPENGYFIQN
TCNNHFNAACGVRCHPGFDLVGSSIILCLPNGLWSGSESYCRVRTCPHL
RQPKHGHISCSTREMLYKTTCLVACDEGYRLEGSDKLTCQGNSQWDGPE
PRCVERHCSTFQMPKDVIISPHNCGKQPAKFGTICYVSCRQGFILSGVK
EMLRCTTSGKWNVGVQAAVCKDVEAPQINCPKDIEAKTLEQQDSANVTW
QIPTAKDNSGEKVSVHVHPAFTPPYLFPIGDVAIVYTATDLSGNQASCI
FHIKVIDAEPPVIDWCRSPPPVQVSEKVHAASWDEPQFSDNSGAELVIT
RSHTQGDLFPQGETIVQYTATDPSGNNRTCDIHIVIKGSPCEIPFTPVN
GDFICTPDNTGVNCTLTCLEGYDFTEGSTDKYYCAYEDGVWKPTYTTEW
PDCAKKRFANHGFKSFEMFYKAARCDDTDLMKKFSEAFETTLGKMVPSF
CSDAEDIDCRLEENLTKKYCLEYNYDYENGFAIGPGGWGAANRLDYSYD
DFLDTVQETATSIGNAKSSRIKRSAPLSDYKIKLIFNITASVPLPDERN
DTLEWENQQRLLQTLETITNKLKRTLNKDPMYSFQLASEILIADSNSLE
TKKASPFCRPGSVLRGRMCVNCPLGTYYNLEHFTCESCRIGSYQDEEGQ
LECKLCPSGMYTEYIHSRNISDCKAQCKQGTYSYSGLETCESCPLGTYQ
PKFGSRSCLSCPENTSTVKRGAVNISACGVPCPEGKFSRSGLMPCHPCP
RDYYQPNAGKAFCLACPFYGTTPFAGSRSITECSSFSSTFSAAEESVVP
PASLGHIKKRHEISSQVFHECFFNPCHNSGTCQQLGRGYVCLCPLGYTG
LKCETDIDECSPLPCLNNGVCKDLVGEFICECPSGYTGQRCEENINECS
SSPCLNKGICVDGVAGYRCTCVKGFVGLHCETEVNECQSNPCLNNAVCE
DQVGGFLCKCPPGFLGTRCGKNVDECLSQPCKNGATCKDGANSFRCLCA
AGFTGSHCELNINECQSNPCRNQATCVDELNSYSCKCQPGFSGKRCETE
QSTGFNLDFEVSGIYGYVMLDGMLPSLHALTCTFWMKSSDDMNYGTPIS
YAVDNGSDNTLLLTDYNGWVLYVNGREKITNCPSVNDGRWHHIAITWTS
ANGIWKVYIDGKLSDGGAGLSVGLPIPGGGALVLGQEQDKKGEGFSPAE
SFVGSISQLNLWDYVLSPQQVKSLATSCPEELSKGNVLAWPDFLSGIVG
KVKIDSKSIFCSDCPRLGGSVPHLRTASEDLKPGSKVNLFCDPGFQLVG
NPVQYCLNQGQWTQPLPHCERISCGVPPPLENGFHSADDFYAGSTVTYQ
CNNGYYLLGDSRMFCTDNGSWNGVSPSCLDVDECAVGSDCSEHASCLNV
DGSYICSCVPPYTGDGKNCAEPIKCKAPGNPENGHSSGEIYTVGAEVTF
SCQEGYQLMGVTKITCLESGEWNHLIPYCKAVSCGKPAIPENGCIEELA
FTFGSKVTYRCNKGYTLAGDKESSCLANSSWSHSPPVCEPVKCSSPENI
NNGKYILSGLTYLSTASYSCDTGYSLQGPSIIECTASGIWDRAPPACHL
VFCGEPPAIKDAVITGNNFTFRNTVTYTCKEGYTLAGLDTIECLADGKW
SRSDQQCLAVSCDEPPIVDHASPETAHRLFGDIAFYYCSDGYSLADNSQ
LLCNAQGKWVPPEGQDMPRCIAHFCEKPPSVSYSILESVSKAKFAAGSV
VSFKCMEGFVLNTSAKIECMRGGQWNPSPMSIQCIPVRCGEPPSIMNGY
ASGSNYSFGAMVAYSCNKGFYIKGEKKSTCEATGQWSSPIPTCHPVSCG
EPPKVENGFLEHTTGRIFESEVRYQCNPGYKSVGSPVFVCQANRHWHSE
SPLMCVPLDCGKPPPIQNGFMKGENFEVGSKVQFFCNEGYELVGDSSWT
CQKSGKWNKKSNPKCMPAKCPEPPLLENQLVLKELTTEVGVVTFSCKEG
HVLQGPSVLKCLPSQQWNDSFPVCKIVLCTPPPLISFGVPIPSSALHFG
STVKYSCVGGFFLRGNSTTLCQPDGTWSSPLPECVPVECPQPEEIPNGI
IDVQGLAYLSTALYTCKPGFELVGNTTTLCGENGHWLGGKPTCKAIECL
KPKEILNGKFSYTDLHYGQTVTYSCNRGFRLEGPSALTCLETGDWDVDA
PSCNAIHCDSPQPIENGFVEGADYSYGAIIIYSCFPGFQVAGHAMQTCE
ESGWSSSIPTCMPIDCGLPPHIDFGDCTKLKDDQGYFEQEDDMMEVPYV
TPHPPYHLGAVAKTWENTKESPATHSSNFLYGTMVSYTCNPGYELLGNP
VLICQEDGTWNGSAPSCISIECDLPTAPENGFLRFTETSMGSAVQYSCK
PGHILAGSDLRLCLENRKWSGASPRCEAISCKKPNPVMNGSIKGSNYTY
LSTLYYECDPGYVLNGTERRTCQDDKNWDEDEPICIPVDCSSPPVSANG
QVRGDEYTFQKEIEYTCNEGFLLEGARSRVCLANGSWSGATPDCVPVRC
ATPPQLANGVTEGLDYGFMKEVTFHCHEGYILHGAPKLTCQSDGNWDAE
IPLCKPVNCGPPEDLAHGFPNGFSFIHGGHIQYQCFPGYKLHGNSSRRC
LSNGSWSGSSPSCLPCRCSTPVIEYGTVNGTDFDCGKAARIQCFKGFKL
LGLSEITCEADGQWSSGFPHCEHTSCGSLPMIPNAFISETSSWKENVIT
YSCRSGYVIQGSSDLICTEKGVWSQPYPVCEPLSCGSPPSVANAVATGE
AHTYESEVKLRCLEGYTMDTDTDTFTCQKDGRWFPERISCSPKKCPLPE
NITHILVHGDDFSVNRQVSVSCAEGYTFEGVNISVCQLDGTWEPPFSDE
SCSPVSCGKPESPEHGFVVGSKYTFESTIIYQCEPGYELEGNRERVCQE
NRQWSGGVAICKETRCETPLEFLNGKADIENRTTGPNVVYSCNRGYSLE
GPSEAHCTENGTWSHPVPLCKPNPCPVPFVIPENALLSEKEFYVDQNVS
IKCREGFLLQGHGIITCNPDETWTQTSAKCEKISCGPPAHVENAIARGV
HYQYGDMITYSCYSGYMLEGFLRSVCLENGTWTSPPICRAVCRFPCQNG
GICQRPNACSCPEGWMGRLCEEPICILPCLNGGRCVAPYQCDCPPGWTG
SRCHTAVCQSPCLNGGKCVRPNRCHCLSSWTGHNCSRKRRTGF.

In some embodiments, the peptide disclosed herein comprises the amino acids positioned at residues 2,637-2,645 of SEQ ID NO: 2. In some embodiments, the peptide comprises SEQ ID NO: 1, and optionally further comprises one or more of: 1-2, 1-3, 1-4, 1-5, 1-6, 2-3, 2-4, 2-5, 2-6, 3-4, 3-5, 3-6, 4-5, 4-6, and 5-6, amino acids from the amino acids set forth at residues 2,631-2,651 of SEQ ID NO: 2. Each possibility represents a separate embodiment of the invention.

In some embodiments, the peptide disclosed herein comprises one or more of: 6-15, 7-15, 8-15, 9-15, 10-15, 11-15, 12-15, 13-15, 14-15, 6-14, 7-14, 8-14, 9-14, 10-14, 11-14, 12-14, 13-14, 6-13, 7-13, 8-13, 9-13, 10-13, 11-13, 12-13, 6-12, 7-12, 8-12, 9-12, 10-12, 11-12, 6-11, 7-11, 8-11, 9-11, 10-11, 6-10, 7-10, 8-10, 9-10, 6-9, 7-9, 8-9, 6-8, 7-8, or 6-7 amino acids from the amino acids set forth at residues 2,631-2,651 of SEQ ID NO: 2. Each possibility represents a separate embodiment of the invention.

In some embodiments, the peptide comprises one or more of: 9-10, 9-11, 9-12, 9-13, 9-14, 9-15, 10-11, 10-12, 10-13, 10-14, 10-15, 11-12, 11-13, 11-14, 11-15, 12-13, 12-14, 12-15, 13-14, 13-15, or 14-15 amino acids from the amino acid sequence: QGYFEQEDDMMEVPYVTPHPP (SEQ ID NO: 3). Each possibility represents a separate embodiment of the invention.

In some embodiments, the peptide comprises 6, 7 or 8 contiguous amino acids from SEQ ID NO: 1. Each possibility represents a separate embodiment of the invention.

In some embodiments, the peptide comprising SEQ ID NO: 1 is an antagonist of the receptor integrin α9β1. The integrin alpha 9 beta 1, or α9β1 receptor, is a multifunctional receptor that is known to interact with a variety of ligands, including vascular cell adhesion molecule 1 (VCAM1), cytotactin tenascin C, osteopontin, nerve growth factor (NGF), brain-derived neurotrophic factor (BDNF) neurotrophin-3 (NT-3), and SVEP1. Integrin subunit alpha 9 (ITGA9) is a protein that in humans (UniProtKB #Q13797) is encoded by the ITGA9 gene (gene ID #3680). Integrin is a heterodimeric integral membrane glycoprotein composed of an alpha chain and a beta chain that mediates cell-cell or cell-matrix adhesion. In some embodiments, α9 subunit, or ITGA9, specifically groups with the R 1 subunit to form the heterodimer integrin α9β1 receptor.

As used herein, the term “an antagonist of a receptor” or “a receptor antagonist”, refers to a receptor ligand that does not activate a biological response itself upon binding to the receptor, but rather blocks or attenuates agonist-mediated response or signaling pathway.

In some embodiments, the peptide disclosed herein comprises a functional analog of SEQ ID NO: 1. The term “analog” as used herein, refers to a peptide that is similar, but not identical, to the peptide disclosed herein. An analog may have deletions or mutations/substitution that result in an amino acids sequence that is different than the amino acid sequence of the peptide. It should be understood that all analogs of the peptide would still be capable of: (a) binding to the α9β1 integrin receptor; and (b) not inducing or promoting signaling via integrin α9β1, or inducing an attenuated or blocked α9β1 signaling, thus referred to as “functional analog(s)”.

The term “attenuated or blocked signaling” refers to a condition in which the signaling being induced by the peptide is reduced by at least: 50%, 60%, 70%, 80%, 90%, 95%, 98%, or 99%, of the level of integrin α9β1 signaling being induced by a control ligand of integrin α9β1. Each possibility represents a separate embodiment of the invention.

Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

In some embodiments, the peptide comprising SEQ ID NO:1, or a functional analog thereof, is incapable of inducing or promoting signaling via integrin α9β1. In other embodiments, the level of integrin α9β1 signaling being induced by the peptide is about 0.0001%-1% of the level of integrin α9β1 signaling being induced by a control ligand of integrin α9β1. In some embodiments, the level of integrin α9β1 signaling being induced by the peptide is one or more of: 0.0001-0.001%, 0.001-0.005%, 0.006-0.01%, 0.01-0.05%, 0.06-0.1%, 0.1-0.5%, and 0.6%-1% of the level of integrin α9β1 signaling being induced by a control ligand. Each possibility represents a separate embodiment of the invention.

As used herein, the term “a control ligand” refers to the SVEP1 polypeptide or the endogenous SVEP1 polypeptide. In some embodiments, a control ligand comprises a mammalian SVEP1. In some embodiments, a control ligand comprises a human SVEP1. In some embodiments, a control ligand comprises the amino acid sequence set forth in SEQ ID NO: 2. In some embodiments, the control ligand comprises a functional analog of SEQ ID NO: 2. As used herein, a “functional analog of SEQ ID NO: 2” refers to a polypeptide that is similar, but not identical, to SEQ ID NO: 2 that still is capable of binding and activating the α9β1 integrin receptor, similarly to the activation by the native SVEP1. In some embodiments, activation of α9β1 integrin receptor can be examined by determination of expression or secretion of pro-inflammatory cytokine, chemokine, or reactive oxygen species, or by determination of phosphorylation of at least one of the signaling components within the signaling pathway of α9β1 integrin receptor.

In some embodiments, the cell disclosed herein expresses the integrin α9β1 receptor. In some embodiments, the cell disclosed herein is a cell of the innate immune system. In some embodiments, the cell is a myeloid cell. In some embodiments, the myeloid cell comprises at least one of: a monocyte, a macrophage, a dendritic cell, a neutrophil, a basophil, an eosinophil, a megakaryocyte, a platelet, and a myeloid-derived suppressor cell (MDSC). In some embodiments, the myeloid cell comprises: a macrophage, a monocyte, or microglia. In some embodiments, the cell disclosed herein comprises a macrophage. In some embodiments the macrophage comprises a monocyte-derived macrophage. In some embodiments, the macrophage comprises a tissue specific macrophage or a tissue-resident macrophage (TRM). TRMs are a heterogeneous population of immune cells that have a tissue-specific and/or a niche-specific function. Examples for TRM include, but not limited to, an adipose tissue macrophage (ATM), giant cells in various tissue such as an osteoclast (found in the bone), an alveolar macrophage (found in the lung), microglial cell (found in the brain), histiocyte (found in the connective tissue), Kupffer cells (found in the liver), or Langerhans cells (LC) (found in the skin). As used herein, the terms “tissue specific macrophage” and “TRM” are interchangeably used. In some embodiments, the cell disclosed herein comprises ATM.

In some embodiments, the macrophage is a bone-marrow derived macrophage.

In some embodiments, the macrophage comprises or is an adipose tissue macrophage.

In some embodiments, a macrophage comprises any descendant cell of a macrophage.

In some embodiments, a descendant cell of a macrophage comprises a giant cell. In some embodiments, a giant cell comprises a multinucleated cell. In some embodiments, a giant cell comprises an inflammatory cell (e.g., involved in inflammation or an inflammatory process). In some embodiments, a giant cell is formed by fusion of a plurality of cells. In some embodiments, a giant cell is a fusion of a plurality of cells. In some embodiments, a giant cell comprises at least 2 nuclei. In some embodiments, a giant cell comprises a plurality of nuclei. In some embodiments, a giant cell excludes a cell comprising a single nucleus. In some embodiments, a cell comprising or having one or a single nucleus is not a giant cell as disclosed herein.

In some embodiments, a descendant cell of a macrophage comprises an osteoclast.

In some embodiments, inhibiting or preventing activation of a myeloid cell comprises reducing at least one parameter selected from: adhesion, migration, cell signaling, expression and/or secretion of at least one pro-inflammatory cytokine, and any combination thereof. In some embodiments, inhibiting or preventing activation of a cell comprises reducing cell adhesion ability. Standard adhesion assays to measure cell binding either to immobilized ligands or to cell monolayers are known in the art, such as in a flat-well microtiter plate under static conditions. Adhesion assay requires several washing steps to separate adherent from nonadherent cells. Adherence or adhesion ability can be examined by counting the number of cells attached to the surface after withdrawal of the medium comprising the cultured cells with or without further several washes. In some embodiments, contacting the myeloid cell with a peptide disclosed herein reduces the adhesion ability of the cell by at least 10%, 15%, 20%, 30%, 40%, 50%, 60%, 70%, 80%, 90%, or 100%, or any value and range therebetween. Each possibility represents a separate embodiment of the invention. In some embodiments, contacting the myeloid cell with a peptide disclosed herein reduces the adhesion ability of the cell by: 10-14%, 15-20%, 21-30%, 31-40%, 40-49%, 50-59%, 60-69%, 70-79%, 80-89%, or 90-100%. Each possibility represents a separate embodiment of the invention.

In some embodiments, inhibiting or preventing activation of a myeloid cell comprises reducing the migration of the cell. A migration assay is performed to determine cell movement to a particular stimulus or chemoattractant. No chemoattractant is used and a cell pathway or trajectory can be tracked. In some embodiments, reduced migration ability comprises reduced accumulative distance of a cell. In some embodiments reduced migration ability comprises reduced speed of a cell. In some embodiments, reduced cell migration comprises reduced by at least: 10%, 15%, 20%, 30%, 40%, 50%, 60%, 70%, 80%, 90%, or 100%, or any value and range therebetween. Each possibility represents a separate embodiment of the invention. In some embodiments, contacting the myeloid cell with a peptide disclosed herein reduces cell migration by: 10-14%, 15-20%, 21-30%, 31-40%, 40-49%, 50-59%, 60-69%, 70-79%, 80-89%, and 90-100%. Each possibility represents a separate embodiment of the invention.

In some embodiments, inhibiting or preventing activation of a myeloid cell comprises reducing cell signaling pathway. In some embodiments, the signaling pathway comprises a pathway involved in an inflammatory response (e.g., immune cell migration or cytokine(s) secretion). In some embodiments, the signaling pathway is selected from: extracellular signal-regulated kinase 1/2/mitogen-activated protein kinase (ERK/MAPK) signaling pathway, jun N-terminal kinase (JNK or JNK/SAPK) signaling pathway, p38 mitogen-activated protein kinase (p38 MAPK) signaling pathway, phosphatidylinositol 3-kinase-Ak strain transforming (PI3K-AKT) signaling pathway, nuclear factor kappa B (NF-kB) signaling pathway, toll-like receptor signaling pathway, Wnt/β-catenin signaling pathway, P53 signaling pathway, Janus kinase-signal transducer and activator of transcription (JAK-STAT) signaling pathway, B cell antigen receptor (BCR) signaling pathway, or any combination thereof. In some embodiments, inhibiting or preventing activation of a cell comprises reducing ERK/MAPK signaling pathway. In some embodiments, the signaling pathway is one of the mammalian MAP kinase cascades comprising: the ERK1 or ERK2 cascade, JNK/SAPK, or p38 pathway. In some embodiments, activated signaling pathway comprises phosphorylation of one of the kinases comprising: ERK1, ERK2, ERK3α, ERK3, ERK3β, ERK1b, JNK1, JNK2, JNK3, p38α, p38β, p38β2, p38γ, p38δ, Mxi, ERK5, ERK7, nemo-like kinase (NLK), male germ cell associated kinase (MAK), MAK-related kinase (MRK), MOK, cyclin dependent kinase like 1 (KKIALRE), or cyclin dependent kinase like 2 (KKIAMRE). In some embodiments, inhibiting or preventing activation comprises reducing or inhibiting phosphorylation of at least one of the components within the ERK/MAPK signaling pathway (e.g., Raf-1, A-Raf, B-Raf, MEK1, MEK2, ERK1 or ERK2). In some embodiments, phosphorylated ERK (p-ERK), comprising p-ERK1 or p-ERK2, or p-ERK1/2, can be used as an end point measurement for the activation of ERK/MAPK signaling pathway. In some embodiments inhibiting or preventing activation of a cell comprises reducing the phosphorylation of ERK (ERK1/2 or p42/44). Methods for detection of cell signaling pathways in general, and phosphorylated ERK, in particular, are well-known in art (e.g., western blot). In some embodiments, reduced cell signaling comprises reduction by at least at least: 10%, 15%, 20%, 30%, 40%, 50%, 60%, 70%, 80%, 90%, or 100%, or any value and range therebetween. Each possibility represents a separate embodiment of the invention. In some embodiments, contacting the myeloid cell with a peptide disclosed herein reduces cell signaling by: 10-14%, 15-20%, 21-30%, 31-40%, 40-49%, 50-59%, 60-69%, 70-79%, 80-89%, or 90-100%. Each possibility represents a separate embodiment of the invention.

In some embodiments, inhibiting or preventing activation of a myeloid cell comprises reducing expression and/or secretion of at least one pro-inflammatory cytokine. In some embodiments, reducing expression and/or secretion of at least one pro-inflammatory cytokine comprises reduced mRNA expression of a gene encoding at least one pro-inflammatory cytokine. In some embodiments, reducing expression and/or secretion of at least one pro-inflammatory cytokine comprises reduced protein expression and/or secretion of at least one pro-inflammatory cytokine. In some embodiments, a pro-inflammatory cytokine comprises: interleukin 1 (IL-1), IL-6, IL-12, tumor necrosis factor alpha (TNF-α), or an y combination thereof. In some embodiments, IL-1 comprises IL-1β. In some embodiments, reduced activation comprises reduced expression or secretion of a chemokine. In some embodiments, the chemokine comprises monocyte chemoattractant protein-1 (MCP-1). In some embodiments, reduced expression or secretion of a cytokine or a chemokine is by at least: 10%, 15%, 20%, 30%, 40%, 50%, 60%, 70%, 80%, 90%, or 100%, or any value and range therebetween. Each possibility represents a separate embodiment of the invention. In some embodiments, contacting the myeloid cell with a peptide disclosed herein reduces cytokine or chemokine expression or secretion by: 10-14%, 15-20%, 21-30%, 31-40%, 40-49%, 50-59%, 60-69%, 70-79%, 80-89%, or 90-100%. Each possibility represents a separate embodiment of the invention.

In some embodiments, a pro-inflammatory cytokine comprises a plurality of pro-inflammatory cytokines. In some embodiments, a pro-inflammatory cytokine comprises a plurality of types of pro-inflammatory cytokines.

In some embodiments, a plurality comprises any integer being equal to or greater than 2.

In some embodiments, inhibiting or preventing activation of a myeloid cell comprises reducing activity of nitric oxide (NO) synthase (NOS). In some embodiments, NO synthase comprises nitric oxide synthase-2 (NOS2). In some embodiments, reduced activity of NO synthase comprises reduced expression levels, e.g., mRNA, protein, or both, of either NOS or NOS2 within a cell. In some embodiments, reduced activity of NO synthase comprises reduced secretion levels of nitrite ion(s). Methods for nitrite detection are known in the art, including Griess assay, which is exemplified herein.

In some embodiments, the myeloid cell disclosed herein expresses the CD11b marker. CD11b is known to be expressed on the surface of a leukocyte, including a monocyte, a neutrophil, a natural killer cell, a granulocyte, or a macrophage. In some embodiments, CD11b is a pan-myeloid marker (i.e., expressed after granulocyte-monocyte progenitor phase in the bone marrow). Methods for identification of a macrophage, as well as distinguishing between TRM and monocyte-derived macrophage are known in the art. In some embodiments, surface markers that can be used to identify a human macrophage include CD11b/Integrin alpha M, CD14, CD68, Fe gamma RIII/CD16, Fe gamma RI/CD64, and CCR5, together with F4/80 in mouse.

In some embodiments, the myeloid cell expresses the CD11c surface marker. In some embodiments, obesity comprises or is characterized by an increase in ATMs displaying the CD11c surface marker, compared to a non-obese control. ATMs are known to be the major contributors to tissue inflammation and insulin resistance in obesity. ATMs in obese mice and humans are known to localize around dead adipocytes, which are more prevalent in obesity, to aggregate and ingest the dying adipocytes. In some embodiments, ATMs of a subject afflicted with obesity secrete increased amounts of proinflammatory cytokines (e.g., TNFα, IL-6, or IL-12), compared to ATMs of a non-obese subject.

In some embodiments, ATMs of a subject afflicted with obesity induce, are involved in, propagate, contribute, any equivalent thereof, or any combination thereof, to the development of insulin resistance and/or type-2 diabetes.

In some embodiments, the myeloid cell comprises M1, M1-like (or a classically activated), or both, macrophage. As used herein, the terms “M1” and “M1-like” are interchangeable, and refer to a pro-inflammatory macrophage, e.g., a macrophage that produces any one of: a pro-inflammatory cytokine, chemokine, reactive oxygen species (ROS), and any combination thereof. In some embodiments, M1 macrophage can be generated by stimulation or activation with a proinflammatory mediator, comprising lipopolysaccharide (LPS) or interferon-γ (IFNγ). In some embodiments, the major subpopulation of ATMs in obesity or other associated metabolic disorders comprises M1 or M1-like macrophages. In some embodiments, a M1-like macrophage secretes high levels of proinflammatory cytokines and/or generates reactive oxygen species through the action of inducible nitric-oxide synthase (NOS). In some embodiments, the macrophage is not an M2 macrophage (or alternatively activated macrophage). M2 macrophage is known to: (i) be generated in vitro by exposure to IL-4 and IL-13, (ii) to secrete low levels of proinflammatory cytokines and (iii) to secrete high levels of anti-inflammatory cytokines. In some embodiments, M1-like macrophage but not M2 macrophage expresses the CD11c surface marker. In some embodiments, CD11c positive M1-like ATMs are the ones that produce the high levels of pro-inflammatory cytokines. In some embodiments, CD11c positive M1-like ATMs are linked to the development of obesity-associated insulin resistance.

In some embodiments, the expression of a cytokine, a chemokine, or an enzyme, refers to protein levels or abundance.

Methods for determining proteins (including peptides or polypeptides) of interest are common and include flow cytometry, immunohistochemical staining of tissue slices or sections, western blot, ELISA, radioimmunoassay (RIA) assays, an antibody microarray, and the like, all of which would be apparent to one of ordinary skill in the art of biochemistry.

In some embodiments, the expression of a cytokine, a chemokine, or an enzyme, refers to gene expression levels (e.g., mRNA).

Numerous methods are known in the art for measuring expression levels of a one or more gene such as by amplification of nucleic acids (e.g., PCR, isothermal methods, rolling circle methods, etc.) or by quantitative in situ hybridization.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts, or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix GenChip technology, or Incyte's microarray technology.

As used herein, the terms “reducing” and “decreasing” are interchangeable and refer to a statistically significant reduction in the expression and/or activity. In one embodiment significant reduction refers to a reduction of at least 10%, or alternatively at least 20%, or alternatively at least 30%, or alternatively at least 40%, or alternatively at least 50%, or alternatively at least 60%, or alternatively at least 70%, or alternatively at least 75%, or alternatively at least 80%, or alternatively at least 85%, or alternatively at least 90%, or alternatively at least 95%, or alternatively at least 97% or alternatively at least 99% reduction in expression and/or activity. Each possibility represents a separate embodiment of the present invention. In some embodiments, reducing or inhibiting is compared to control. In some embodiments, a control is a healthy control. In some embodiments, a control is a non-treated control. In some embodiments, a control is of the same subject in two different time points, e.g., before and after treatment.

In some embodiments, the myeloid cell comprises a myeloid cell of a subject. In some embodiments, the myeloid cell of a subject expresses the α9β1 integrin. In some embodiments, the myeloid cell of a subject comprises a monocyte, a macrophage, a dendritic cell, a neutrophil, a basophil, an eosinophil, a megakaryocyte, a platelet, a myeloid-derived suppressor cell (MDSC), or any combination thereof. In some embodiments, the myeloid cell is a macrophage comprising monocyte-derived macrophage or TRM of a subject. In some embodiments, the myeloid cell comprises a TRM of a subject. In some embodiments, the TRM is M1, or M1-like macrophage associated with an inflammatory disease or disorder in a subject.

In some embodiments, the cell is a cell of a subject in need of treatment, and the contacting comprises administering to the subject a therapeutically effective amount of the peptide comprising SEQ ID NO: 1, or a functional analog thereof. In some embodiments, the subject is afflicted with an inflammatory or an autoimmune disease or disorder. In some embodiments, the inflammatory or autoimmune disease or disorder comprises an imbalance of M1-like and M2-like macrophages towards a M1-like macrophages. Inflammatory conditions or autoimmune diseases associated with elevated levels of M1, or M1-like macrophages are known in the art. Several examples for diseases associate with dysregulated or elevated levels of M1-like macrophages comprise asthma, chronic obstructive pulmonary disease, atherosclerosis, or rheumatoid arthritis, as disclosed in Atri C et al. Role of Human Macrophage Polarization in Inflammation during Infectious Diseases. Int J Mol Sci. 2018; 19:1801, herby incorporated by reference in its entirety.

In some embodiments, there is provided a method for treating or preventing an integrin α9β1-related disease, or disorder, in a subject in need thereof, comprising administering to the subject a therapeutically effective amount of the peptide disclosed herein, comprising (SEQ ID NO: 1). In some embodiments, the α9β1-related disease or disorder is or comprises or is characterized by a macrophage cell expressing or harboring an integrin α9β1.

In some embodiments, the disease or disorder is selected from: cancer, hepatic fibrosis, bone or joint destruction, a metabolic bone disease, and a cytokine storm condition. In some embodiments, cancer comprises prostate cancer, melanoma, breast cancer, colon cancer, rhabdomyosarcoma, squamous cell carcinoma (SCC) or hepatocellular carcinoma. In some embodiments, bone or joint destruction comprises osteoporosis, bone fracture, Paget's disease, osteoarthritis, rheumatoid arthritis, gout, or bursitis. In some embodiments, a metabolic bone disease is caused by abnormality of a mineral such as calcium or phosphorus, or abnormality of vitamin D. In some embodiments, a cytokine storm condition (or hypercytokinemia) is caused by a viral infection. In some embodiments, a viral infection comprises H1N1 influenza, H5N1 influenza, SARS-CoV-1, SARS-CoV-2, influenza B, parainfluenza virus, Ebola, Epstein-Barr virus, cytomegalovirus, or group A streptococcus. In some embodiments the cytokine storm can be caused by a non-infectious condition. In some embodiments, non-infectious condition comprises graft-versus-host (GVH) disease.

In some embodiments, the inflammatory disease or disorder comprises an imbalance of M1-like and M2-like macrophages within ATMs. In some embodiments, inflammatory disease or disorder comprises a metabolic syndrome, disease, disorder, or condition. As used herein, the term a “metabolic syndrome, disease, disorder, or condition” refers to any disease or disorder characterized by excess abdominal fat, hypertension, abnormal fasting plasma glucose level or insulin resistance, high triglyceride levels, low high-density lipoprotein (HDL) cholesterol level, and any combination thereof. In some embodiments, the metabolic syndrome disorders which can be treated according to the present invention are diverse and will be easily understood by the skilled artisan. Without any limitation, mentioned are obesity, pre-diabetes, diabetes, hyperglycemia, diabetic dyslipidemia, hyperlipidemia, hypertriglyceridemia, hyper-fattyacidemia, hypercholesterolemia, hyperinsulinemia, insulin-resistance, or insulin-resistance related. High risks of metabolic syndrome disease include, but are not limited to, obstructive sleep apnea, nonalcoholic steatohepatitis, chronic kidney disease, polycystic ovary syndrome and low plasma testosterone, erectile dysfunction, or both. In some embodiments, the inflammatory disease or disorder comprises a cardiometabolic disease (CMD). Examples of CMDs include cardiovascular disease (CVD), diabetes mellitus and chronic renal failure. Examples for CVDs includes coronary heart disease, stroke, or transient ischemic attack (TIA), peripheral arterial disease, or aortic disease. In some embodiments, an inflammatory disease or disorder comprises adipose tissue inflammation. In some embodiments, an inflammatory disease or disorder comprises bone marrow inflammation.

In some embodiments, there is provided a pharmaceutical composition comprising a peptide comprising the amino acid sequence: EDDMMEVPY (SEQ ID NO: 1), or a functional analog, and a carrier.

In some embodiments, the pharmaceutical composition is for use in the treatment or prevention of an inflammatory disease or disorder, as disclosed herein.

In some embodiments, the pharmaceutical composition is for use in the treatment or prevention of a metabolic disease or disorder, as disclosed herein.

In some embodiments, there is provided a method for treating or preventing an inflammatory disease or disorder, in a subject in need thereof. In some embodiments, there is provided a method for treating or preventing a metabolic syndrome, comprising obesity, insulin resistance or diabetic mellitus, in a subject in need thereof. In some embodiments, the method comprising administering to the subject a therapeutically effective amount of a pharmaceutical composition comprising a peptide comprising the amino acid sequence: EDDMMEVPY (SEQ ID NO: 1), or a functional analog. In some embodiments, the peptide comprises or consists of 9 to 15 amino acids. In some embodiments, the peptide is of 9 to 15 amino acids.

The term “therapeutically effective amount” refers to the concentration of the peptide that is normalized to body weight (BW) and is effective to treat a disease or disorder in a mammal. The term “a therapeutically effective amount” refers to an amount effective, at dosages and for periods of time necessary, to achieve the desired therapeutic or prophylactic result. A physician of ordinary skill can readily determine and prescribe the effective amount of the bioactive agent required. The exact dosage form and regimen would be determined by the physician according to the patient's condition. The dosage administered will be dependent upon the age, health, and weight of the recipient, kind of concurrent treatment, if any, frequency of treatment, and the nature of the effect desired.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal. The dosage administered will be dependent upon the age, health, and weight of the recipient, kind of concurrent treatment, if any, frequency of treatment, and the nature of the effect desired.

In some embodiments, there is provided a method for treating or preventing a metabolic disease or disorder, in a subject in need thereof, the method comprising administering to the subject a therapeutically effective amount of a pharmaceutical composition comprising a peptide comprising the amino acid sequence: EDDMMEVPY (SEQ ID NO: 1), and wherein the peptide comprises or is of 9 to 15 amino acids, thereby treating or preventing a metabolic disease or disorder, in the subject.

In some embodiments, the metabolic disease or disorder comprises obesity. In some embodiments, the metabolic disease or disorder comprises pre-diabetes. In some embodiments, the metabolic disease or disorder comprises diabetes. In some embodiments, the metabolic disease or disorder comprises insulin-resistance. In some embodiments, diabetes comprises type-2 diabetes. In some embodiments, diabetes comprises type-1 diabetes.

In some embodiments, the metabolic disease or disorder comprises hyperglycemia. In some embodiments, the metabolic disease or disorder comprises diabetic dyslipidemia. In some embodiments, the metabolic disease or disorder comprises hyperlipidemia. In some embodiments, the metabolic disease or disorder comprises hypertriglyceridemia. In some embodiments, the metabolic disease or disorder comprises hyper-fattyacidemia. In some embodiments, the metabolic disease or disorder comprises hypercholesterolemia. In some embodiments, the metabolic disease or disorder comprises hyperinsulinemia. In some embodiments, the metabolic disease or disorder comprises hypertension.

In some embodiments, treating or preventing comprises reducing weight of or in the subject. In some embodiments, treating or preventing comprises reducing adipose tissue volume or weight in the subject. In some embodiments, treating or preventing comprises increasing insulin sensitivity of the subject. In some embodiments, treating or preventing comprises reducing glucose level in the subject. In some embodiments, treating or preventing comprises reducing the number of inflammatory cells in the subject. In some embodiments, treating or preventing comprises reducing the number of leukocytes in the subject. In some embodiments, treating or preventing comprises reducing the number of macrophages in the subject. In some embodiments, treating or preventing comprises reducing the number of ATMs in the subject. In some embodiments, treating or preventing comprises reducing the level of a pro-inflammatory cytokine, a chemokine, or both, in the subject. In some embodiments, at least one of: the number of inflammatory cells, the number of leukocytes, the number of macrophages, the number of ATMs, the level of a pro-inflammatory cytokine or a chemokine, or any combination thereof, is reduced in an adipose tissue, in or of the subject. In some embodiments, the treating or preventing comprises reducing the number or abundance of pro-inflammatory ATMs in the subject.

In some embodiments, administering comprises local administering. In some embodiments, administering comprises injecting into a fatty tissue, in the subject. In some embodiments, administering comprises subcutaneously injecting, in the subject.

In some embodiments, the route of administration of the pharmaceutical composition disclosed herein comprises an intravenous route, an intramuscular route, a subcutaneous route, or an oral delivery route. The route of administration of the pharmaceutical composition will depend on the disease or condition in need of treatment. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate compositions it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. In some embodiments, the composition of the invention comprising oral delivery. In some embodiments, the composition of the invention comprises an oral composition. In some embodiments, the composition of the invention further comprises orally acceptable carrier, excipient, or a diluent.

As used herein, the terms “subject” or “individual” or “animal” or “patient” or “mammal” refers to any subject, particularly a mammalian subject, for whom therapy is desired, for example, a human.

In some embodiments, the pharmaceutical composition comprises a pharmaceutically acceptable carrier, adjuvant, or excipient.

As used herein, the term “carrier”, “adjuvant”, or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18^{th} Ed., Mack Publishing Co., Easton, Pa. (1990); and Remington: The Science and Practice of Pharmacy, 21^{st} Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the term “about” when combined with a value refers to plus and minus 10% of the reference value. For example, a length of about 1000 nanometers (nm) refers to a length of 1000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Animals

The SVEP1 knockout (KO) experiments involved breeding male C57bl/6 SVEP1 heterozygous knockout mice with wildtype C57bl/6 mice to generate the SVEP1^{+/-} mice. Littermate controls were used in all experiments. In the peptide experiment, six-week-old male C57bl/6J mice were fed with chow or high-fat diet for twelve weeks to generate a diet-induced obese mice model. For glucose- and insulin-tolerance tests, male mice at 14 and 24 weeks were fasted for 12 hours, followed by intraperitoneal injection of glucose (2 g/kg) and insulin (0.75 U/kg). Glucometer readings were taken from tail vein blood samples. The mice were kept in a conventional facility with 12 hours of light/dark cycles and were provided with water ad libitum. Animal care and experiments were in accordance with the guidelines of the IACUC Approval (TAU-MD-IL-2212-178-4, and TAU-MD-IL-2212-179-4).

The epididymal visceral adipose tissues (VAT) and livers were dissected from mice and used as fresh or frozen tissues. For the peptide ex vivo analysis, epididymal adipose tissue was minced and subsequently incubated in a trans-well system for eight to sixteen hours with 30 μM of the peptide and LPS (100 ng/ml) in a conditioned medium.

### Peptide

Unlabeled and fluorescein isothiocyanate (FITC) labeled peptide (P-FITC) of 9 amino acids (EDDMMEVPY) were synthesized and solubilized in DMSO for the subsequent assays.

Isolation of the Stromal Vascular Fraction from Tissue

Tissues were minced and finely homogenized in HBSS solution (Biological Industries). Following this, collagenase solution was employed for tissue digestion at 37° C. for one hour with agitation. The digested tissue was filtered through a 100 μm cell strainer and centrifuged. Mature adipocytes were discarded, and the pellet containing SVF cells was collected after suspension in red blood cell lysis buffer. For culturing purposes, the SVF fraction was seeded, and the medium was replaced after 24 hours post-seeding. The attached Adipose-derived stem and progenitor cells were cultured in a growth medium, which consisted of DMEM (Gibco) supplemented with 10% fetal bovine serum (Biological Industries), 1% L-glutamine (Biological Industries), 1% penicillin-streptomycin (Sigma), 0.5% 4-(2-hydroxyethyl)-1-piperazine-ethanesulfonic acid (HEPES; Biological Industries), This medium is referred as growth medium and was used as the basic medium for all other cell types.

### Bone Marrow-Derived Macrophages (BMDM) Cultures and RAW264.7 Cells

Bone marrow was isolated from femurs and pelvises of 6-12 weeks old C57bl/6J mice. Cells were then filtered, isolated, and cultured in a growth medium supplemented with 10% CMG-conditioned media; the medium was changed twice a week.

Mouse RAW264.7 cells (American Type Culture Collection) were also cultured in a growth medium, which was changed twice a week.

### Decellularization of Adipose-Derived Stem and Progenitor Cells

APSCs were washed with PBSX1 two times and then were frozen at −80° C. and thawed. The cultures were then incubated with a decellularization solution containing PBS containing 0.5% and NH4OH (20 mM). The ECM was then treated with DNase (100 g/ml) and was washed once more.

### Adhesion and Migration Assays

Suspended RAW264.7 cells and BMDM cells were treated with varying peptide concentrations for 30 minutes. The cells were seeded on 96-well plates and incubated for five minutes. Next, the cells were thoroughly washed, fixated, and stained for DAPI in order to detect the number of adhered cells in each well. The data was relative to DMSO-treated cells.

Adhered RAW264.7 cells were treated with the peptide, and cell migration was tracked using time-lapse images over a three-hour period. Migration paths were calculated using ImageJ's manual cell tracker plugin. Accumulative distance and average speed were determined.

All related imaging analysis was performed using EVOS FL Auto 2 microscope (Invitrogen).

### Immunofluorescence Staining and Fluorescent Imaging for Tissue and Cells

Fresh isolated epididymal adipose tissues were used for whole-mount staining. Isolated tissue was fixated in 1% paraformaldehyde for one hour, washed, and blocked with a blocking buffer, PBS-0.3T with 5% normal goat serum, then incubated overnight with primary monoclonal antibody ITGA9, F4/80, Prelipin 1 (Santa Cruz), and SVEP1 polyclonal antibody. Secondary antibodies used were Alexa Fluor 555 anti-Mouse IgG1 (Invitrogen), Alexa Fluor 488 anti-Mouse IgG1 (Invitrogen), Cy3-anti-rabbit and 647anti-rat (Jackson ImmunoResearch Laboratories) and Fluoroshield™ mounting medium containing DAPI. Images were acquired by a confocal microscope (Leica SP8; Leica, Wetzlar, Germany).

RAW264.7 cells were fixed with a 4% paraformaldehyde solution, permeabilized with 0.5% Triton in 1% TBST, and then blocked with a blocking solution (1% TBST containing 1-2% normal goat serum and 1% BSA). The cells were incubated overnight with primary ITGA9 (Santa Cruz) and F4/80 (Santa Cruz) antibodies, washed, and incubated with secondary antibodies, Cy3-anti-mouse (Jackson ImmunoResearch Laboratories), Alexa Fluor 555 anti-Mouse IgG1 (Invitrogen), and Alexa Fluor 488 anti-Mouse IgG2b (Invitrogen) for one additional hour. The stained coverslips were mounted on slides with Fluoroshield™ mounting medium containing 4′, 6-diamidino-2-phenylindole (DAPI). Images were acquired by a confocal microscope (Leica SP8; Leica, Wetzlar, Germany) or EVOS FL Auto 2 microscope (Invitrogen).

### SiRNA of RAW264.7 Cells

For siRNA assays, RAW264.7 cells were transfected by using Avalanche®-Everyday Transfection Reagent (Ezbio systems) with siSCR (Santa Cruz) and siITGA9 (Santa Cruz) in DMEM. The transfection solution was added to the cells for 48 hours, and the cells were examined after an additional 48 hours (96 hours in total).

### LPS Activation Assays

Lipopolysaccharide (LPS; Sigma) 100 nM was added to the cells' medium for 30 min or up to 8 hours, depend ending on the assay. For the western blot analysis, the cells were treated with 100 nM of LPS and 30 μM of peptide before the lysate extraction. In the LPS cytometric analysis, cells were treated with 100 nM of LPS for 4 hours, and in the last hour, the FITC-peptide was added. For the nitric oxide levels secreted to culture media (analyzed by Griess assay, Promega) and for the pro-inflammatory gene expression by qPCR, the cells were treated with 100 nM of LPS and 30 μM of the peptide for 8 hours.

### Flow Cytometry Analysis of Isolated Tissue and RAW264.7 Cells

Isolated SVF cells from adipose tissue were suspended in a staining buffer; PBS with 2% serum and 0.1% NaN3 for 15 min and then stained with Anti-CD45-APC, Anti-F4/80-FITC, Anti-CD11b-PE/Cy5 (Bio-gems), CD11c-APC/Cy7, MHCII-PE/Cy7 and Ly6C-Pacific blue (Biolegend).

RAW264.7 cells were incubated in PBS with 2% serum, 0.1% NaN3, and 1 mM EDTA for 20 minutes, collected, and centrifuged at 1800 rpm for 5 min. Next, the cells were suspended in a staining buffer; PBS with 2% serum and 0.1% NaN3 for 15 min on ice. The cells were stained for ITGA9-PE (R&D technologies).

Cells were also analyzed for the FITC-labeled peptide, which was added to the suspension of RAW264.7 cells in doses of 100 nM, 1 μM, 3 μM, 10 μM, 30 μM, 100 μM, and 300 μM, for one hour. For the time curve binding assay, 3 μM of the FITC-peptide were incubated with cells for 1, 5, 15, 60, 180, and 360 minutes before and analyzed on flow cytometry. For the propidium iodide (PI) assay, cells were incubated with different doses of the peptide for eight and 24 hours, with 10% DMSO serving as a positive control; the cells were then suspended, stained with PI, and analyzed with flow cytometry.

All labeled cells were analyzed by Cytoflex5l and Kaluza software (Beckman Coulter instrument).

### XTT Analysis

Cells were incubated with 1 μM, 3 μM, 10 μM, 30 μM, 100 μM, and 300 μM of the peptide for three, eight, and twenty-four hours. The XTT solution (Cell Proliferation Kit (XTT based); Biological industries) was added to the wells for three hours, and the absorbance (450 nm minus 650 nm) was then measured with a microplate Spectra MAX M5 plate reader (Spectra MAX M5; Molecular Devices).

### Immunoblotting

The procedures and analyses were performed according to the standard protocols (protocol-online.net). Cells were harvested from cultures, washed with ice-cold PBS, and lysed in 50 mM Tris pH 7.5, 150 mM NaCl buffer containing one mM EDTA, 1% NP-40, and protease inhibitors: [phenylmethylsulfonyl fluoride (PMSF), 1 mM; 1-chloro-3-tosylamido-4-phenyl-2-butanone, TPCK, 10 μg/ml; aprotinin, 10 μg/ml (Sigma-Aldrich)]. The tissues were isolated and homogenized in the same lysis buffer. Protein concentration was determined with BCA Protein Assay Kit (Pierce). Samples were re-suspended in Laemmli buffer, separated on 7.5% SDS-PAGE gel, and transferred to nitrocellulose. After blocking, the membranes were incubated overnight with a primary antibody anti-ERK/anti-pERK (Cell signaling technology). For detection, the second antibody used was Peroxidase Anti-Rabbit IgG (Jackson Immuno Research), and the peroxidase signal was detected with chemiluminescent substrate (Pierce) read on Fusion FX7 (Vilber).

RNA Isolation and qPCR

Total RNA was extracted from RAW264.7 cells and tissues (Bio Tri RNA; Bio-Lab Ltd.) and reverse transcribed to cDNA using the High-Capacity cDNA Reverse Transcription Kit (Applied Biosystems). Transcripts levels were measured with SYBR green (Applied Biosystems) using STEPONE plus system (Thermo Fisher Scientific). All data were normalized to Actin by the delta-delta CT method.

### Bioinformatics Analysis

Bioinformatics analysis for the scRNAseq experiment of Edmont et al., 2022, was done with the “Seurat” R package. For the tree trajectory analysis, all steps were performed using the “CytoTree” R package. FCS data from the adipose tissue macrophages experiment was preprocessed and converted to a “Cyt” object, a clustering analysis was then performed to achieve a dimensionality reduction by SOM method, and the cell trajectory tree was generated using t-distributed stochastic neighbor embedding (tSNE) method.

### Protein Binding Site and Interaction Prediction

Modeling was performed using Google Colab, a publicly available version of ColabFold. The structures were predicted using the PDB100 database. For the ITGA9:ITGB1 complex, the respective 30-620 and 30-482 first amino acids were used for prediction (NLDPQ . . . FERNC: QTDEN . . . SEGIP). MSA search was done by MMseqs2 on the UniRef100 database, and the top-ranked relaxed model was used for further analysis. Besides the peptide, the binding site peptides used were TNC: AEIDGIEL, VCAM-1: IDSPL, EMILIN-1: PEGLENKP, and OPN: SVVYGLR. Scannet was used to determine protein binding sites hotspots in the integrin α9β1 complex. Pymol was used for visualization and alignment scores.

### Statistical Analysis

Statistical analyses were analyzed by GraphPad Prism v.8.1.1. Results are presented as means±SEM. All results were tested for normal distribution by the Kolmogorov-Smirnov test, and outliers were identified using the ROUT method. Statistical differences comparing the mean values were tested using two-tailed, unpaired t-tests or one way-ANOVA where appropriate. Values that were not normally distributed were tested using Mann-Whitney or Kruskal-Wallis (for three or more groups), followed by Dunn's post-test for multiple comparisons. A value of p<0.05 was considered statistically significant.

### Example 1

### Integrin 9β1 is Expressed in Adipose Tissue Macrophages

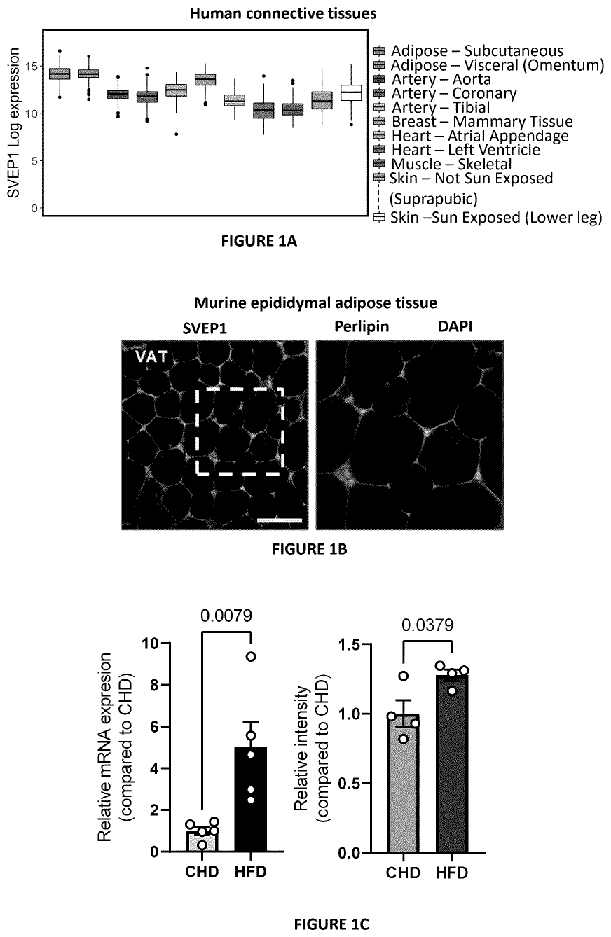

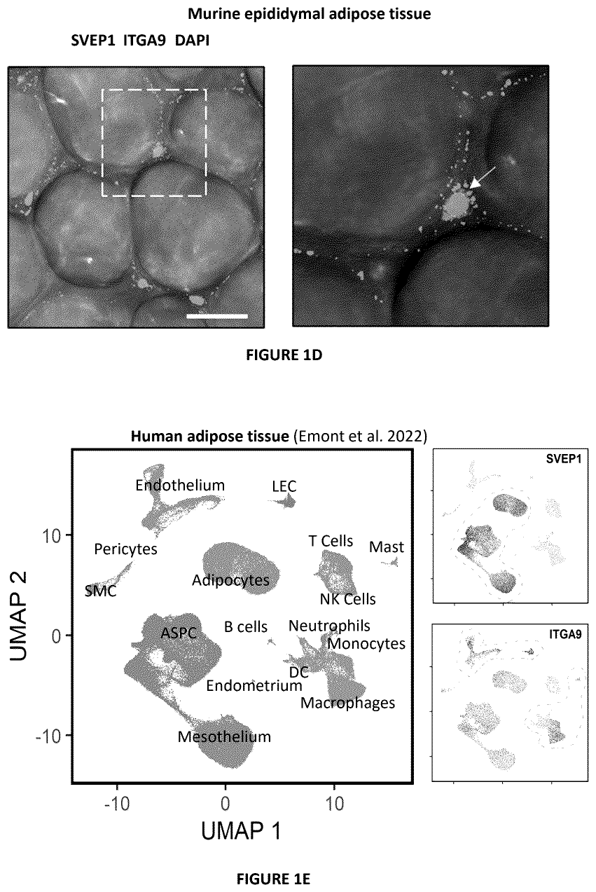

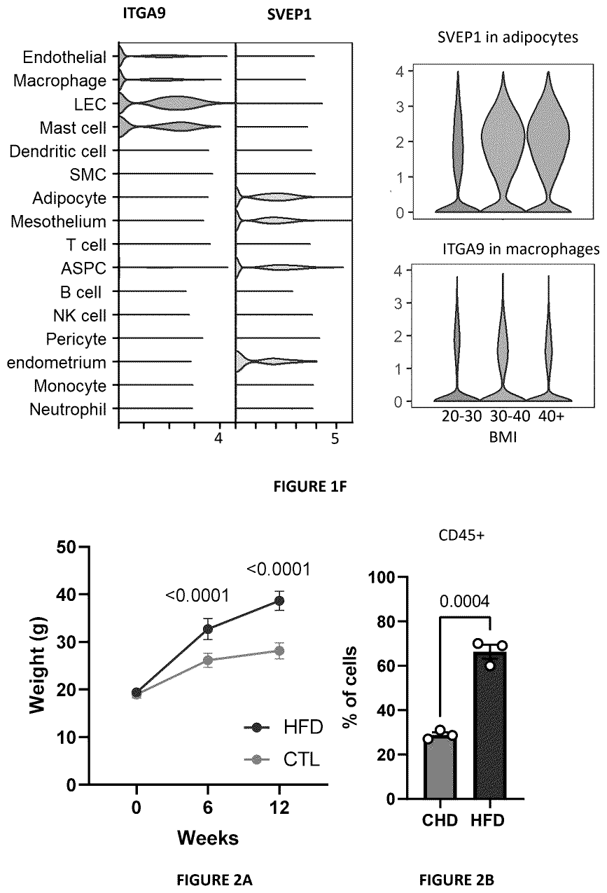

SVEP1 is a 400 kDa multidomain protein that acts as a cell adhesion molecule and is mainly located in mesenchymal cells. Emerging evidence indicates that SVEP1 is also involved in adipose tissue function with its upregulation in subcutaneous and visceral fat depots of obese patients. In vivo mice model revealed that heterozygous KO of SVEP1 results in decreased fat mass and body mass. Moreover, a coding variation in the SVEP1 gene was correlated with an increased prevalence of diabetes, hypertension, and coronary artery disease. Integrin α9β1 is a transmembrane receptor and a known receptor for SVEP1. The inventors first sought to characterize the expression patterns of SVEP1 and integrin α9β1 in adipose tissue. First, the inventors examined the expression patterns of SVEP1 in human connective tissues from GteX (FIG. 1A), where it was highly expressed in adipose tissues. A wholemount staining of murine visceral adipose tissue showed that it is mainly expressed in adipocytes where it colocalized with Perilipin and in the extracellular matrix (FIG. 1B). When the inventors compared SVEP1's expression in HFD and CHD-fed mice both mRNA and protein levels were upregulated in the obese mice (FIGS. 1C-1D). SVEP1 was then co-stained with its receptor, integrin α9β1, to examine their localization patterns.

Interestingly, Integrin was not colocalized with SVEP1 and was found on non-adipocytes in intracellular junctions, which might suggest a potential interaction pattern (FIG. 1E). Next, single-cell data from human adipose tissues was used to determine ITGA9's expression patterns. As shown in FIG. 1F, Integrin α9 (ITGA9) is expressed in endothelial cells, mast cells, and macrophages, in contrast to SVEP1, which is expressed mainly in the mesenchymal populations. A subclusters analysis of the prominent populations in the tissue revealed that SVEP1 is highly expressed in high-BMI adipocytes and that ITGA9 is found in all the macrophages subpopulations, with a slight upregulation in higher BMIs (FIG. 1G). ITGA9 heterodimerizes exclusively with Integrin β1 (ITGB1), which is ubiquitously expressed in the different clusters, and therefore, ITGA9 expression serves as a proper alternative for Integrin α9β1.

### Example 2

### Integrin α9β1 in Adipose Tissue Subpopulations

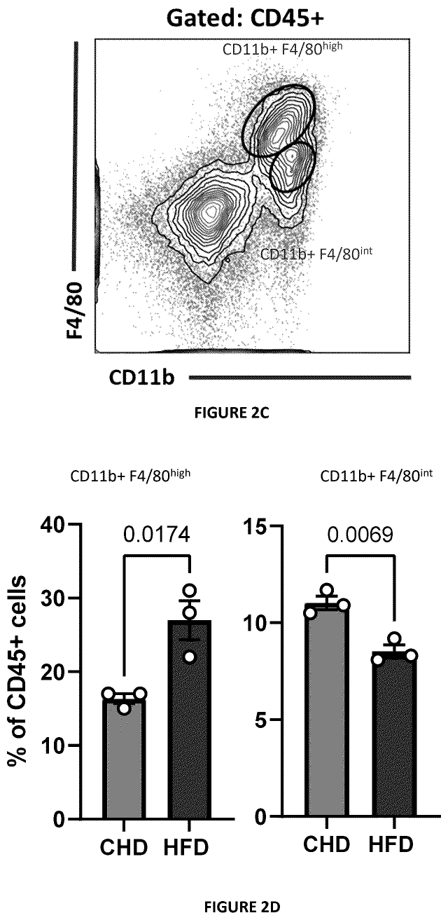

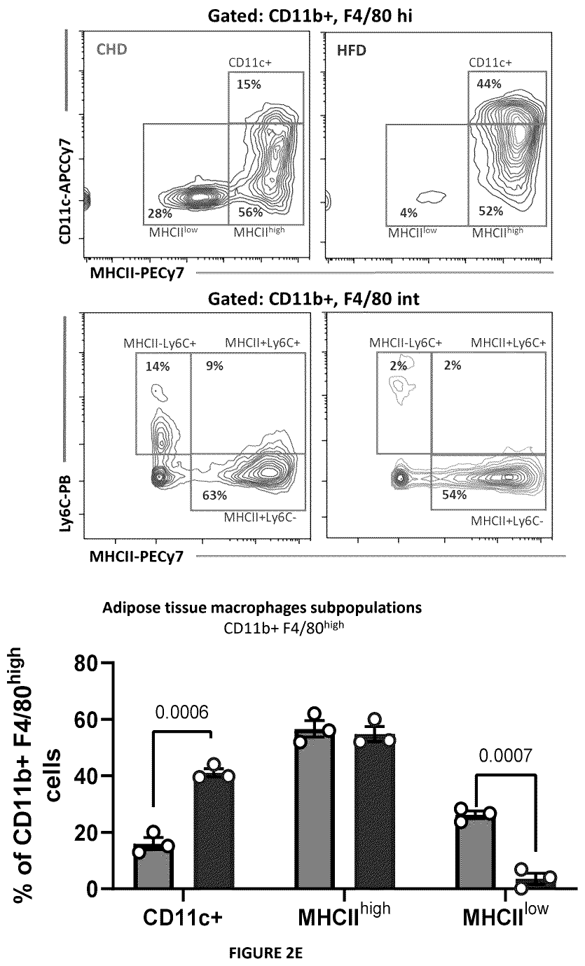

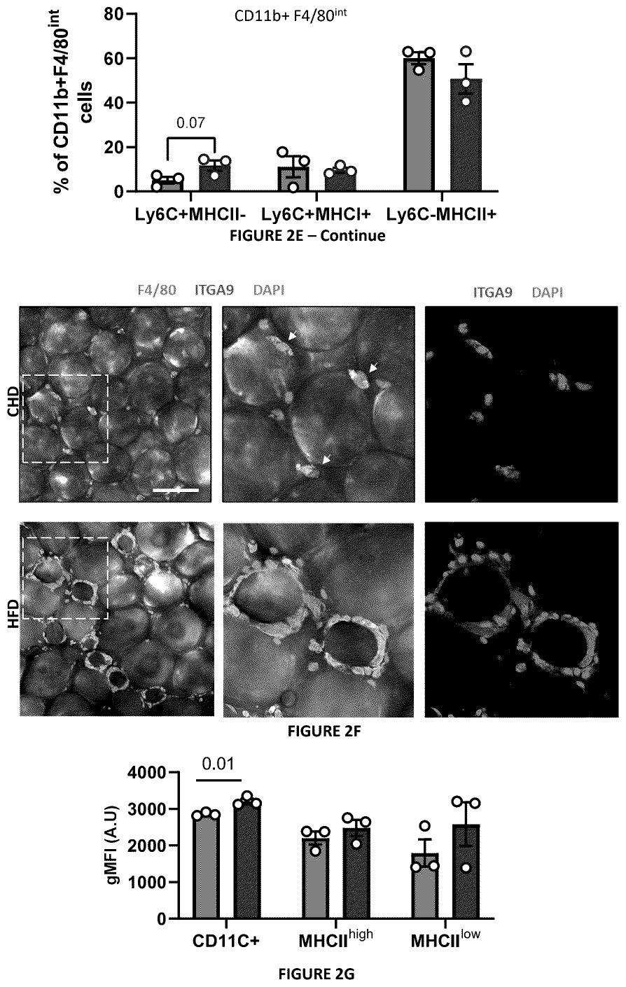

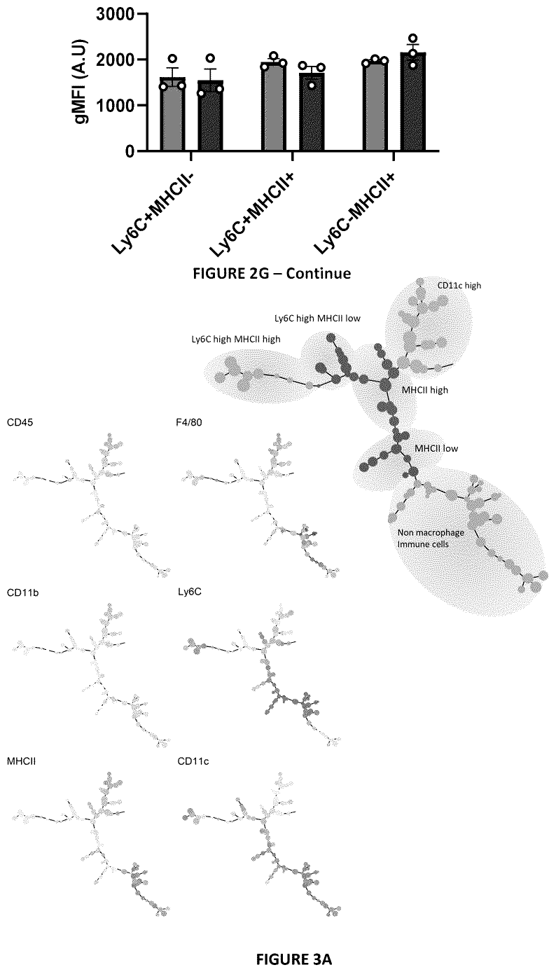

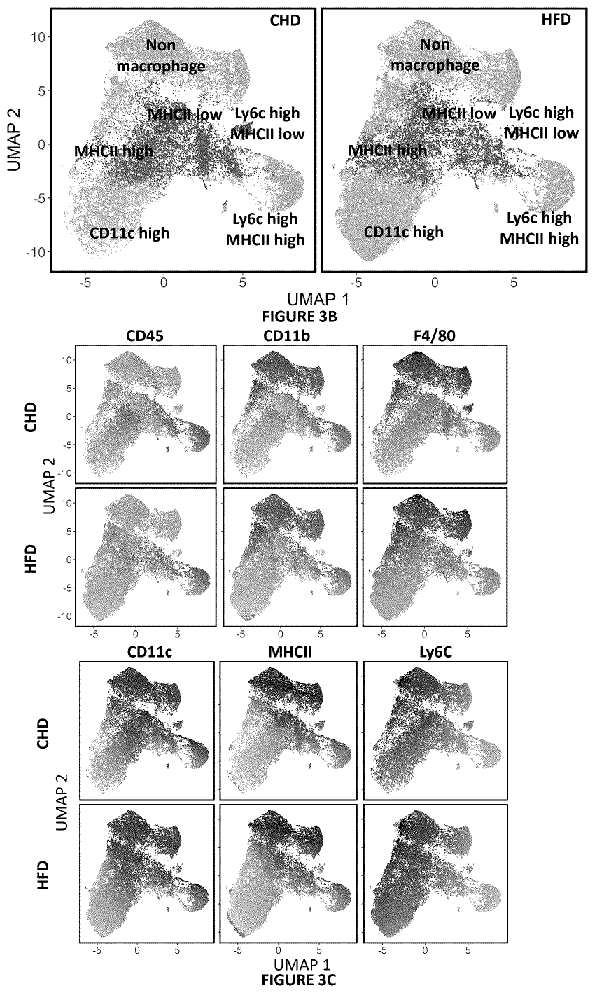

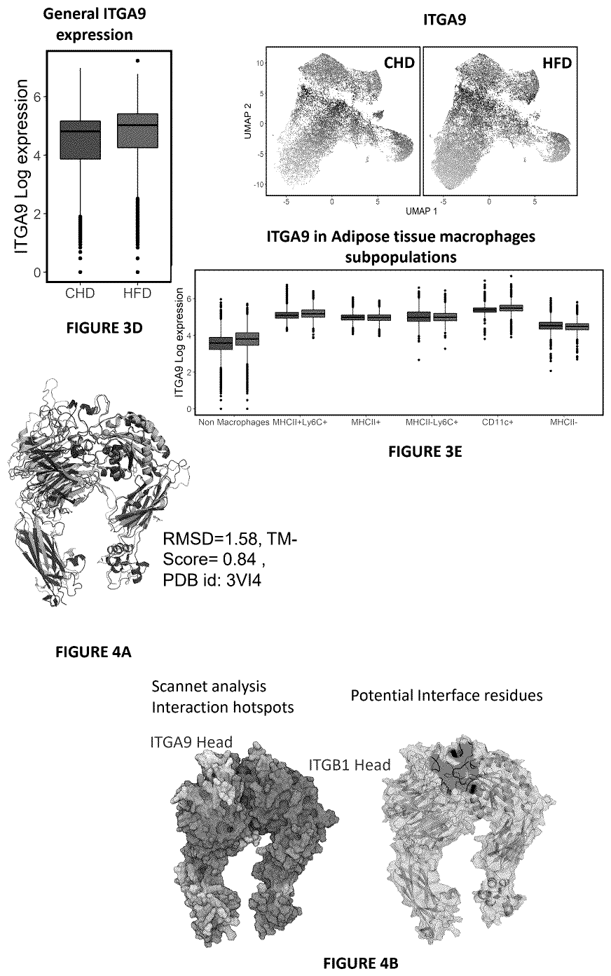

The inventors sought to establish an adipose tissue macrophage (ATM) subpopulation analysis. Mice were fed high-fat or normal diets for 12 weeks (FIG. 2A) to examine the differential ATM subpopulation patterns in response to a nutritional challenge. After 12 weeks of HFD, the stromal vascular fractions of epididymal adipose tissues were isolated and examined. Unsurprisingly, HFD samples had a significantly higher number of immune cells (CD45+), which indicates the possible infiltration of these cells into the HFD tissues (FIG. 2B). F4/80 and CD11b were then used to identify the ATMs together with CD45. To subdivide the ATMs, F4/80 high and intermediate expressing cells (F4/80^{high}, F4/80^{int}) were regarded as two distinct subpopulations. F4/80^{high}, CD11b+, CD45+ cells, which are considered as ATMs, were found in higher percentage in the obese mice, while the F4/80^{int}, CD11b+, CD45+ monocytic macrophages were higher in the CHD mice (FIG. 2C). The F4/80^{high}, CD11b+, and CD45+ ATMs were further subdivided based on their MHCII and CD11c levels. The CD11c− (MHCII^{+/-}) populations were shown to be a primary resident ATM subpopulation in lean mice, while the CD11c+ cells were significantly upregulated in obese mice. Monocytes (F4/80^{int}, Ly6C+, MHCII−) and monocyte-derived macrophages (F4/80^{int}, Ly6C−, MHCII+) were also analyzed with no significant changes witnessed (FIG. 2D). To validate the ability to cluster and identify the subpopulations, the inventors constructed a Uniform Manifold Approximation and Projection for Dimension Reduction (UMAP) analysis and generated a tree-shaped trajectory that identifies the cellular population based on the current flow cytometry results (FIG. 3A). Based on the current tree-shaped trajectory clusters analysis, the inventors then generated a low-dimensional representation of the current results that corresponds with the different subpopulations. As was also shown in FIG. 2D, HFD samples had more CD11c high and fewer MHCII low ATMs, which indicates a possible turnover of these two populations (FIGS. 3B-3C). According to the current marker expression plots, the different subpopulations are distinct and identifiable by the expression of the markers' combinations effectively.

After establishing the ATMs subpopulation analysis, the inventors investigated the expression pattern of Integrin α9β1 in murine adipose tissue. Immunofluorescence staining of ITGA9 and F4/80 revealed a high colocalization pattern in HFD and chow-fed mice (FIG. 2E). General normalized expression analysis showed higher levels of ITGA9 in HFD mice. When examined in the ATM subpopulations, it was primarily expressed in macrophages with a general upregulation in obese conditions and specifically in the CD11c+ and MHC+ subsets, where ITGA9 had significantly higher levels in obese mice (FIGS. 2F and 3E-3F). These results support that Integrin α9β1 is found in ATM and indicates a possible role in specific obese-related macrophage subsets that may interact with SVEP1 in an adipocyte-macrophage axis.

### Example 3

### Characterization of the Peptide's Binding Properties

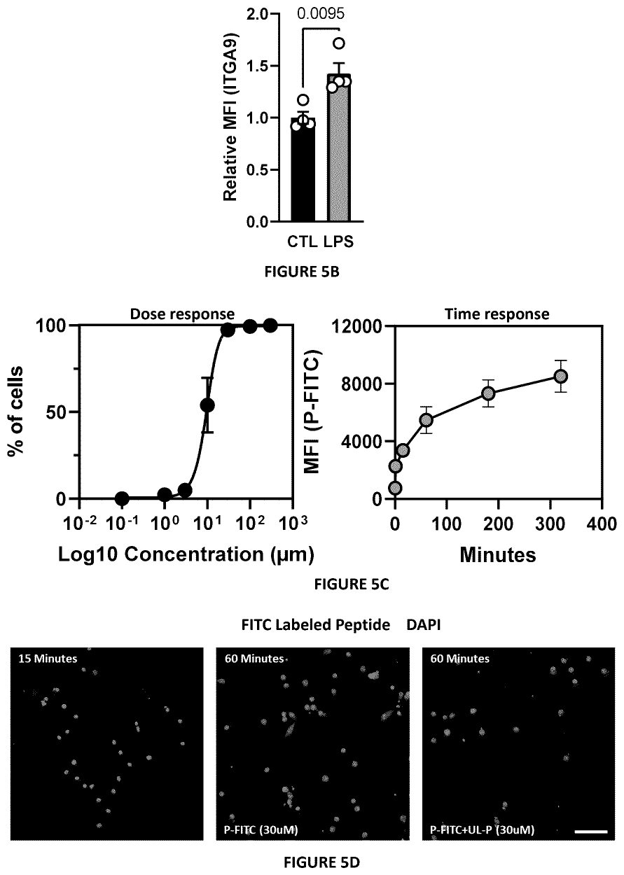

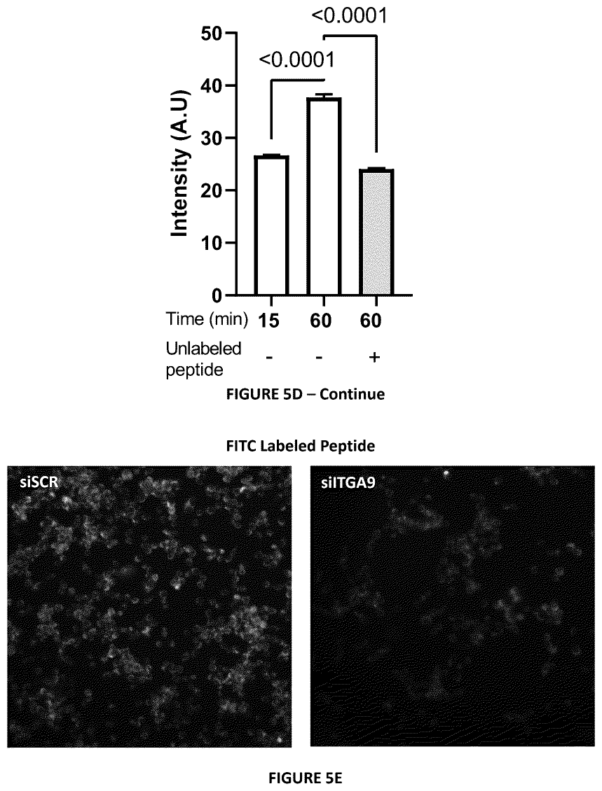

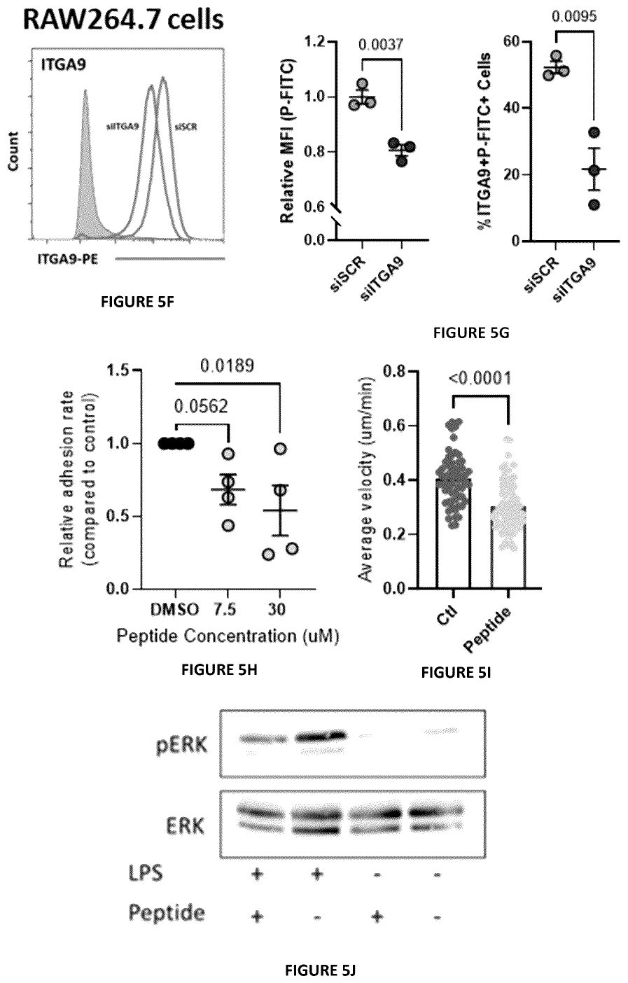

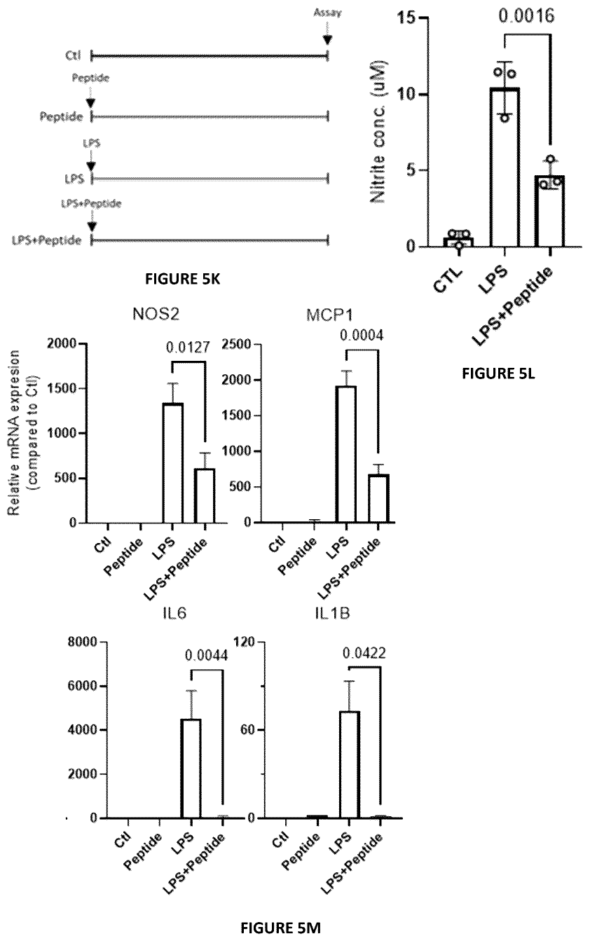

In a previous study, a 9 amino-acid long sequence, “EDDMMEVPY”, was identified as the binding site of SVEP1 to integrin α9β1. To examine the effect of the peptide in vitro, RAW264.7 cells, a murine macrophage-like cell line, was chosen due to their similarity to macrophages, adhesive and migratory capabilities, and their expression of integrin α9β1, as shown in the confocal staining of ITGA9 and F4/80, with a seeming membranal expression of both (FIG. 5G). The inventors also examined ITGA9 levels as a response to an inflammatory stimulus that showed an upregulation of ITGA9 in LPS-induced cells (FIG. 5B). To characterize the binding properties of the peptide, a fluorescein isothiocyanate (FITC) labeled peptide was generated. The inventors first performed a dose-dependent uptake test, where the RAW264.7 cells were incubated with increasing peptide concentrations for half an hour and then measured in a flow cytometry assay. The increasing levels of the peptide resulted in a higher percentage of positive cells in a dose-related manner. Next, the cells were pretreated with the peptide for different time durations and were also measured by flow cytometry analysis. The peptide bonded relatively quickly, with 50% of max mean fluorescence intensity presented after 15 minutes and stabilization after three hours (FIG. 5C). Fluorescent live microscopy was used to further assess the binding properties of the peptide. Raw264.7 cells were incubated with the peptide for 15 and 60 minutes; the inventors also co-incubated the FITC labeled peptide with an unlabeled peptide to evaluate competitive binding. As shown in FIG. 5D, intensity levels were increased by 35% after 60 minutes compared to 15 minutes of incubation and decreased by a similar percentage when co-incubated with the unlabeled peptide. Next, the inventors used a small interfering RNA to knockdown ITGA9 in RAW264.7 cells to analyze the binding specificity of the peptide, validation of the KD efficiency was measured by flow cytometry that showed a 50% reduction in the membranal levels of ITGA9 in KD cells (FIG. 5E). Fluorescence imaging and flow cytometry analysis of KD and scrambled cells displayed a reduction in the FITC-peptide binding, with lower intensity levels in siITGA9 cells (FIGS. 5F-5G). As a result, the percentage of ITGA9+FITC-peptide+ cells was significantly lower in KD cells (FIG. 5G). The inventors then sought to assess the function of the peptide in RAW 264.7 cells by examining its ability to inhibit adhesion and migration. To determine adhesion, suspended cells were incubated with the peptide for half an hour before seeding. The cells incubated with the peptide had a lower adhesion rate than the control cells, with the 30 μM treatment displaying a 50% decrease in adhesion compared to the control (FIG. 5H). The inventors then performed a migration assay to assess the effect on motility in three hours. The average speed of the treated cells was significantly decreased as for their accumulated distance as well (FIG. 5I). This data indicates that the SVEP1-based peptide potentially attenuates the Integrin pathway and thus impedes migration in adhesion in RAW264.7 cells. To examine the molecular pathway by which the peptide affects the Integrin axis, the inventors have performed a western blot analysis of ERK and phosphorylated ERK (pERK). The cells were also treated with LPS to examine the peptide's impact on activated cells. The pERK levels of the peptide treatment cells decreased compared to the control cells.

Interestingly, even in the presence of LPS, an inducer of the ERK pathway, the peptide-treated cells had lower levels of ERK phosphorylation (FIG. 5J). Due to the peptide's capability to inhibit LPS-induced ERK phosphorylation, the inventors strove to explore its potential inhibitory effect on the inflammatory activation process. As shown by both the Griess assay and the qPCR analysis of pro-inflammatory cytokines (FIGS. 5I-5M), coincubation of the peptide with LPS repressed the inflammatory response dramatically, with reductions in both nitrites levels and mRNA expression levels of several cytokines, including IL1b and IL6. Taken together, these results suggest that SVEP1-based peptide has the potential to hinder integrin α9β1 activity and substantially reduce adhesion, motility, and activation of macrophages in vitro.

### Example 4

### Integrin α9β1-SVEP1-Based Peptide Spatial Binding Prediction

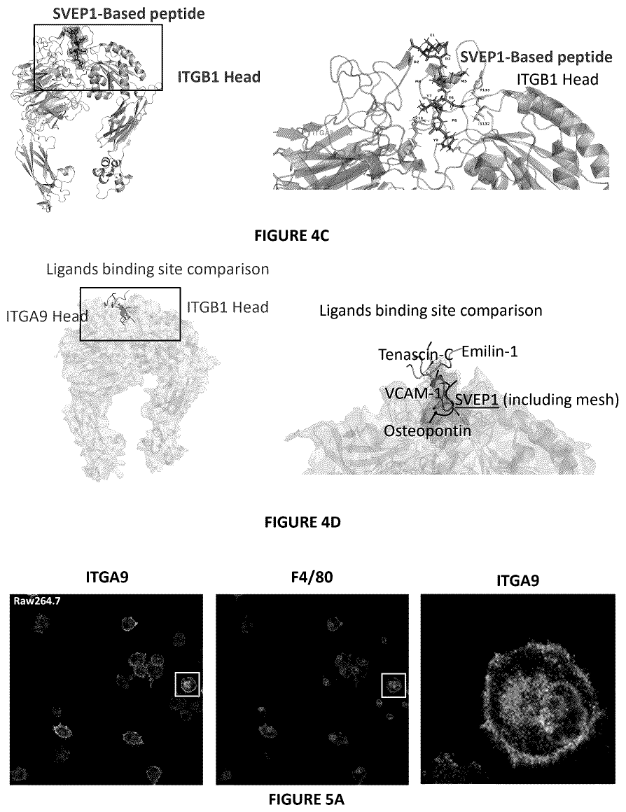

The alignment of the current integrin α9β1 Alphafold prediction with the PDB structure of integrin α5β1 (3VI4) revealed a high structural similarity, with a TM-score of 0.84, which provides a strong foundation for subsequent analyses (FIG. 4A). Scannet analysis, which predicts possible protein binding sites from structures of the predicted model, highlighted a specific protein binding site on integrin α9β1. Notably, this identified hotspot corresponded precisely to the binding site of the SVEP1-based peptide (FIG. 4B). This observation underscores the potential importance of this region in ligand recognition and binding. Further examination of the SVEP1-based peptide and integrin α9β1 interaction elucidated critical amino acids at the binding interface. In particular, two amino acids within the peptide were shown to be directly involved in interactions with the receptor (Glu:6 and Mte:5), substantiating the importance of these residues in mediating the binding affinity and specificity of the interaction. This insight into the atomic-level interactions enhances current understanding of the peptide's mode of binding and its potential functional relevancy (FIG. 4C).

Interestingly, the binding spot identified for the SVEP1-based peptide was found to overlap with the binding sites of other known ligands of integrin α9β1, including Tenascin-C, Emilin-1, Osteopontin, and VCAM-1. This convergence of binding sites among various ligands implies a commonality in their interaction mechanisms and reinforces the significance of this binding domain in integrin α9β1's function. Moreover, this observation suggests the intriguing possibility that the SVEP1-based peptide might not only competitively inhibit the interaction between SVEP1 and integrin α9β1 but also potentially interfere with the binding of other ligands, thereby offering a broader range of inhibitory effects (FIG. 4D).

### Example 5

### The Peptide does not Affect the Metabolic Activity and Cell Death Rate in Macrophages

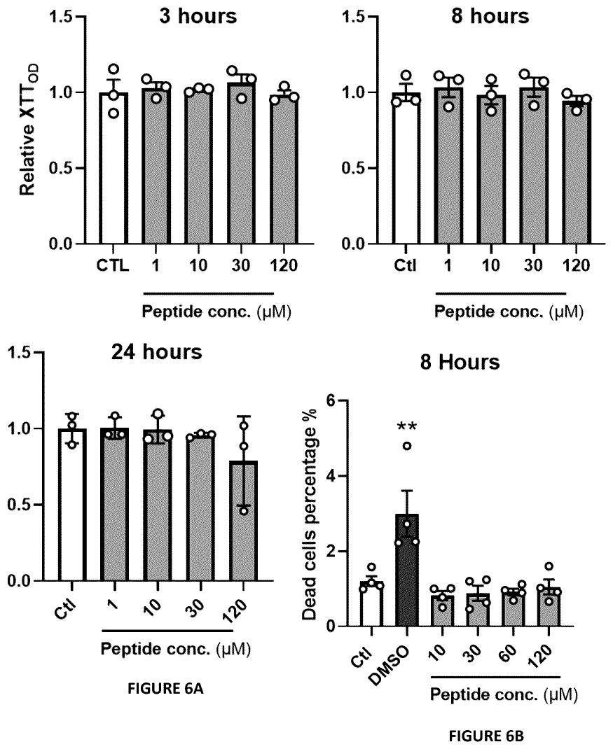

To investigate the effects of the SVEP1-based peptide on the metabolic and proliferative capacity of macrophage cells, RAW264.7 cells were subjected to varying concentrations of the peptide (1, 10, 30, 120 μM). The cells' metabolic activity was assessed using XTT after 3, 8, and 24 hours of peptide treatment. Importantly, no significant differences were observed among the groups even after 3, 8, and 24 hours of exposure (FIG. 6A). To further evaluate cell viability, propidium iodide (PI) staining was conducted after 8 and 24 hours of peptide treatment. Markedly, the fraction of PI-positive cells remained unchanged compared to the untreated group, even at higher peptide concentrations, indicating that the peptide does not induce cell death through apoptosis (FIG. 6B). These findings provide evidence of the peptide's safety profile and its potential suitability for use in macrophage cells.

### Example 6

### The Peptide Effectively Inhibits Adhesion and Inflammation Activation in Bone Marrow Derived Macrophages (BMDM)

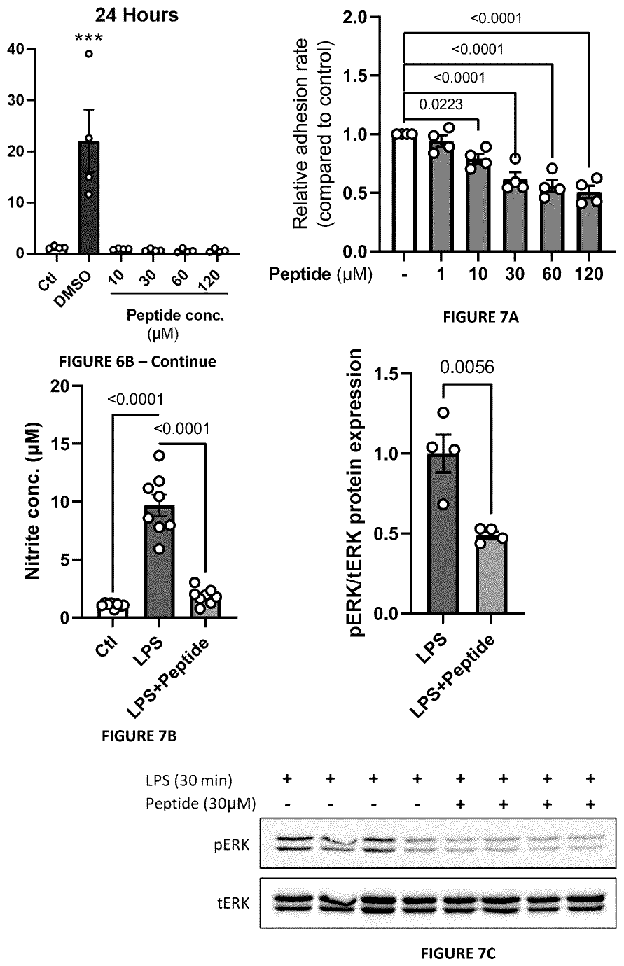

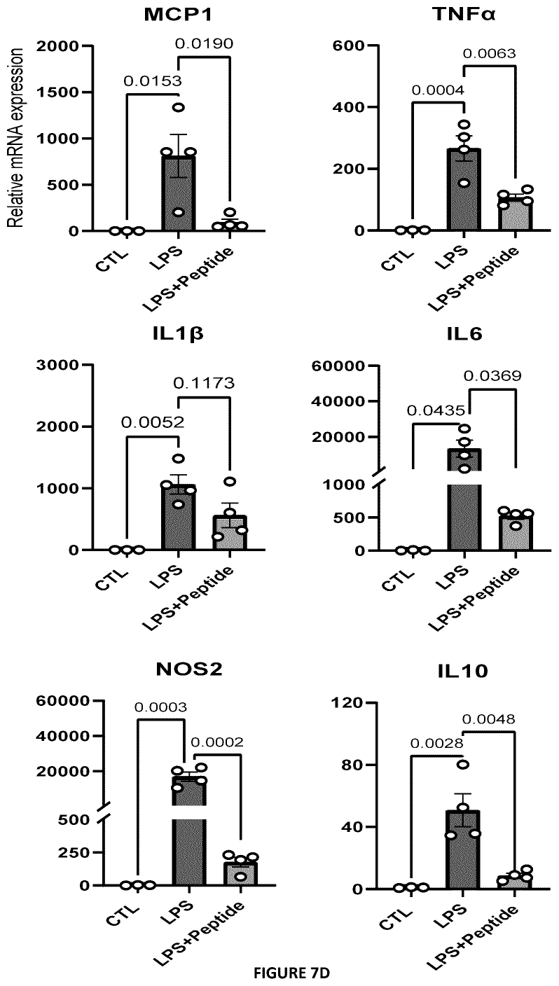

In order to assess the functionality of the peptide in bone marrow-derived macrophages (BMDM) obtained from murine bone marrow, the inventors conducted a series of experiments. The first set of experiments focused on cell adhesion, which involved incubating suspended BMDM with the peptide for 30 minutes before seeding them at different concentrations. To mimic BMDM-extracellular matrix interaction, the inventors performed adhesion experiments on a decellularized extracellular matrix derived from mesenchymal cells. Intriguingly, the BMDM treated with the peptide exhibited a significant reduction of 40% in adhesion compared to control cells at a concentration of 30 μM (FIG. 7A). Moreover, higher concentrations of the peptide resulted in even greater reductions, reaching up to 50%. These findings shed light on the potential of the SVEP1-based peptide to attenuate the Integrin role and therefore its pathway, thereby reducing the adhesion capability of bone marrow-derived macrophages.

Next, the current study aimed to determine whether the peptide could attenuate inflammation in BMDM in the same manner as in RAW264.7 cells. To assess this, the inventors employed a Griess assay, which measures nitrite levels in the media and serves as an indicator of proinflammatory macrophage activation. BMDM were incubated with LPS, either with or without the peptide, for a duration of eight hours before quantifying nitrite levels. Remarkably, the group co-incubated with the peptide, and LPS displayed significantly lower levels of nitrites compared to the group treated with LPS alone. This finding suggests the potential of the peptide to suppress the inflammatory response in BMDM (FIG. 7B).

Motivated by this experiment, the inventors delved deeper into the potential inhibitory effect of the peptide on the inflammatory activation process. To gain insights into the underlying molecular pathway influenced by the peptide within the Integrin axis, the inventors conducted a western blot analysis of ERK and pERK. Additionally, the inventors treated the BMDM with LPS to examine the peptide's impact on activated macrophages. Interestingly, the peptide-treated BMDM exhibited lower levels of ERK phosphorylation compared to the control cells (FIG. 7C). This intriguing observation indicates that the peptide possesses the ability to inhibit LPS-induced ERK phosphorylation. Furthermore, a qPCR analysis of pro-inflammatory cytokines provided further affirmation of these findings, as co-incubation of the peptide with LPS significantly repressed the inflammatory response, leading to reduced mRNA expression levels of various cytokines, including NOS2, IL6, and TNFα (FIG. 7D).

Taken together, these findings provide compelling evidence that the SVEP1-based peptide holds a potential to impede the activity of integrin α9β1 and substantially reduce adhesion and activation of bone marrow-derived macrophages. The utilization of these cells in the current study significantly strengthens the significance of the current results in the context of macrophage function. This research opens up new avenues for future investigations and highlights the therapeutic implications of targeting SVEP1 in modulating macrophage-mediated processes.

### Example 7

### The Peptide Inhibits Osteoclastogenesis

Integrin-dependent signaling pathways are known to play a role in bone resorption, and antibodies are used to inhibit this pathway in vitro and in vivo. The integrin role in osteoclast function relies on the RGD-domain, and RGD-containing peptides were shown to raise cytosolic calcium in osteoclasts. Integrin's role in the formation of giant cells/osteoclasts also localize to the sealing zone of actively resorbing osteoclasts, suggesting that they play a role in linking the adhesion of osteoclasts to the bone matrix with the cytoskeletal organization and the polarization and activation of these cells for bone resorption. Integrins are known to mediate cell-matrix and cell-cell interactions.

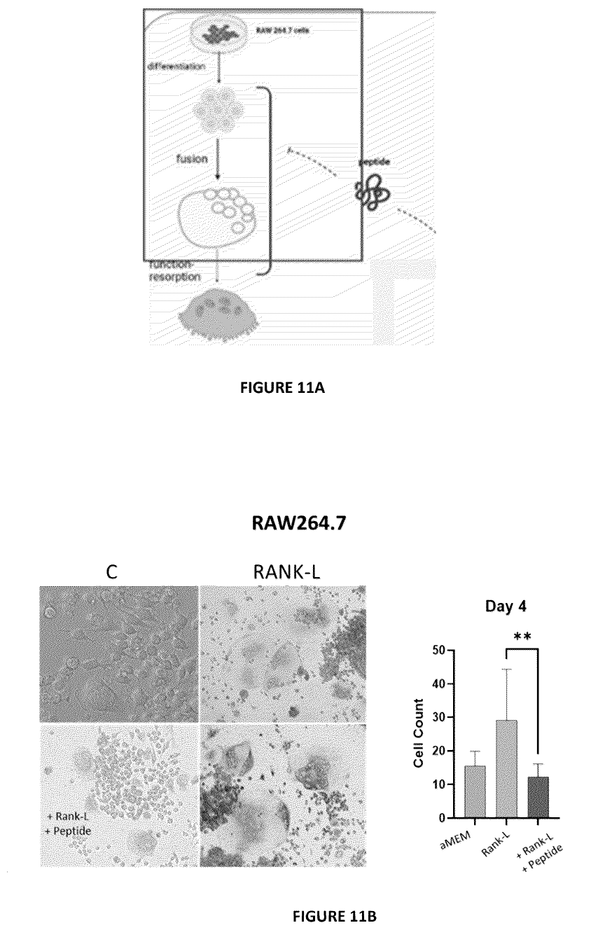

The macrophages expressing the integrin α9β1 (FIGS. 1D-1F) are osteoclast precursors, and therefore, the peptide was further used to study the inhibition of osteoclastogenesis and to analyze its effect on the osteoclast formation. The osteoclasts are formed from mononuclear precursors to multinuclear giant cells, which is induced by Rank-ligand, LPS, TNFα, etc., some of which are demonstrated in FIGS. 11A-11B.

The inventors analyzed the peptide attenuation on osteoclast formation in RAW264.7 cells and imaging analysis along with TRAP staining was used to follow the giant cells multi-nucleated are TRAP⁺, a predominant enzyme for these cells' functionality (FIG. 11B). BMDM were also used, and both presented the same pathway of osteoclastogenesis. Remarkably, the cells co-incubated with Rank-L and the peptide displayed significantly lower levels of osteoclasts formation (FIG. 11B) and lower TRAP+ expression.

### Example 8

### SVEP1, an Extracellular Matrix Protein Highly Expressed in Adipose Tissue, Effects Glucose Metabolism

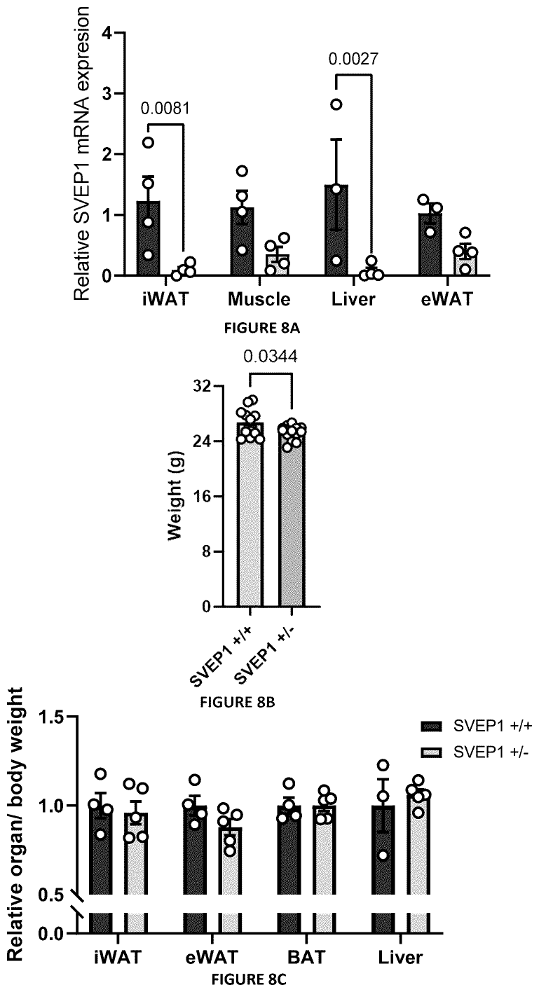

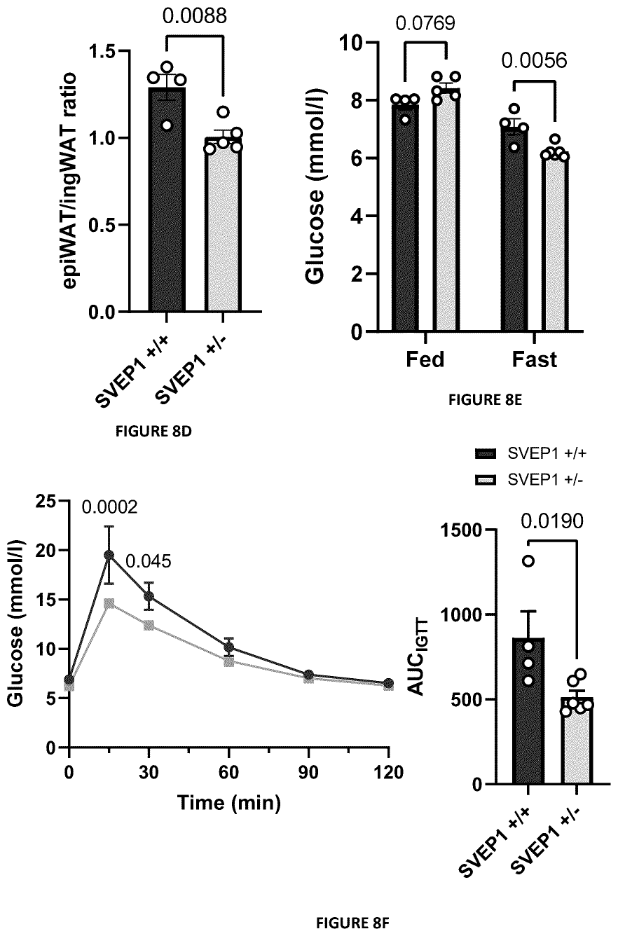

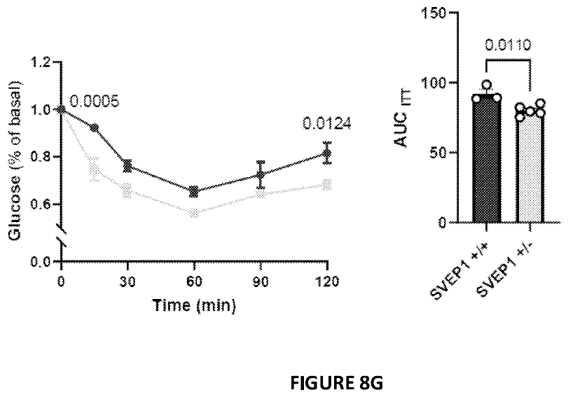

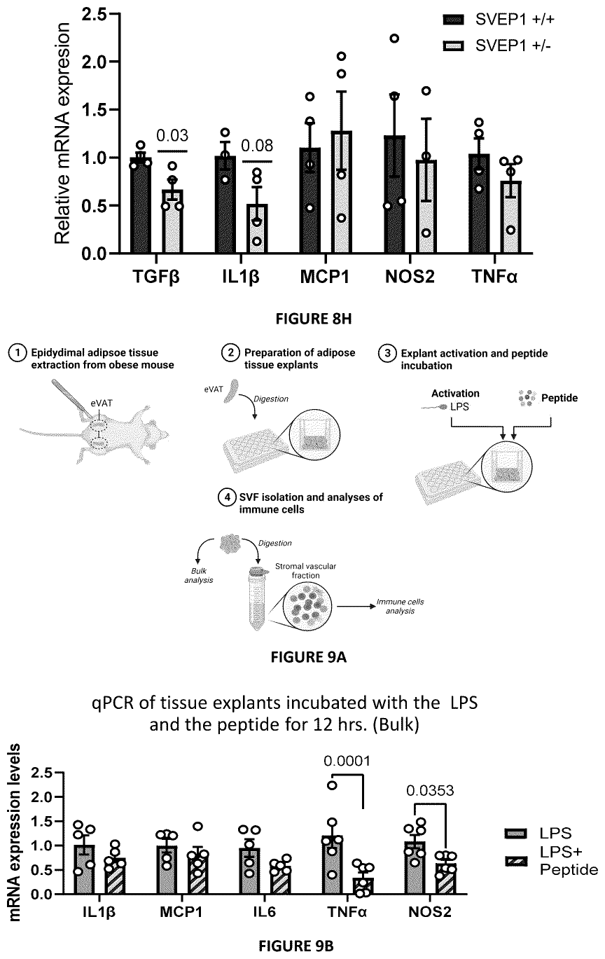

To gain insights into the physiological significance of SVEP1, the molecule upon which the peptide is based, the inventors utilized SVEP1 heterozygous mice (SVEP1^{+/-}) (FIG. 8A). Previous studies have demonstrated the influence of this mouse model on systemic metabolism (Meehan et al., 2017); however, a comprehensive analysis of adipose tissue metabolism remains unexplored. Notably, SVEP1 knockout (KO) mice exhibited lower body weight with a 6% reduction compared to WT mice after 14 weeks (FIG. 8B). While the KO mice exhibited adipose tissue weights comparable to the wild-type group, they displayed a significantly more favorable subcutaneous to visceral adipose tissue ratio (FIGS. 8C-8D). This observation potentially explains the effect of SVEP1 on glucose metabolism, as previous associations between SVEP1 and glucose metabolism and diabetes have been reported.

Therefore, the current objective was to assess glucose homeostasis and insulin sensitivity in the SVEP1 KO mice. Fasting glucose levels were markedly reduced by 30% in the KO mice group (FIG. 8E). Intraperitoneal glucose and insulin tolerance tests unveiled enhanced glucose tolerance and insulin sensitivity in the SVEP1 KO mice, implying a potential metabolic role for SVEP1 in regulating glucose homeostasis (FIGS. 8F-8G). Furthermore, a quantitative polymerase chain reaction (qPCR) analysis of proinflammatory genes revealed alterations in TGFβ mRNA levels, suggesting a possible involvement of SVEP1 in tissue fibrosis and its regulation of inflammation levels and metabolism (FIG. 8H).

Taken together, these findings underscore the role of SVEP1 in adipose tissue and metabolism, highlighting the potential therapeutic intervention by inhibiting SVEP1's function. This comprehensive understanding of SVEP1's impact opens new avenues for future research and offers prospects for targeted interventions aimed at modulating its effects.

### Example 9

### The Peptide's Effect on Inflamed Adipose Tissue Explants (Ex Vivo)

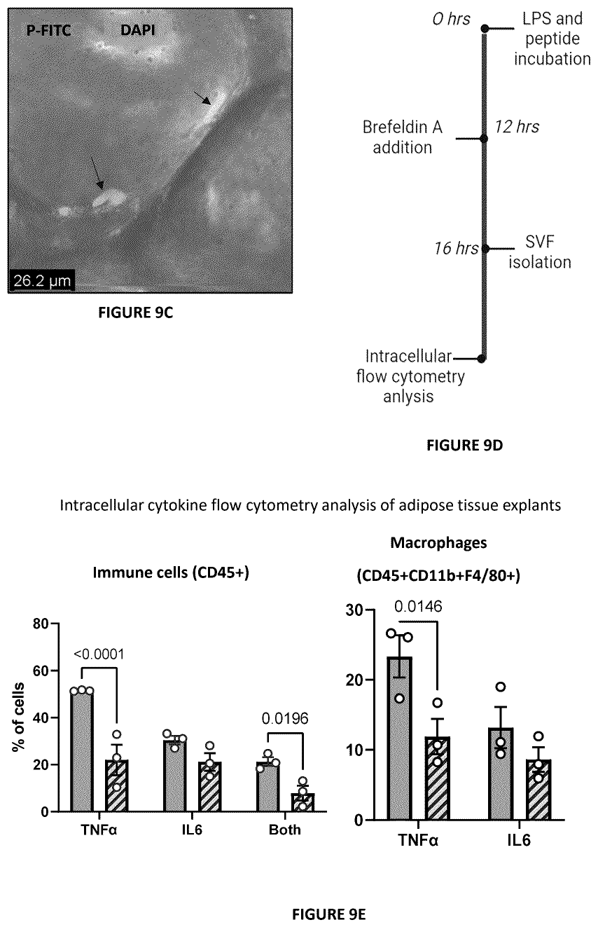

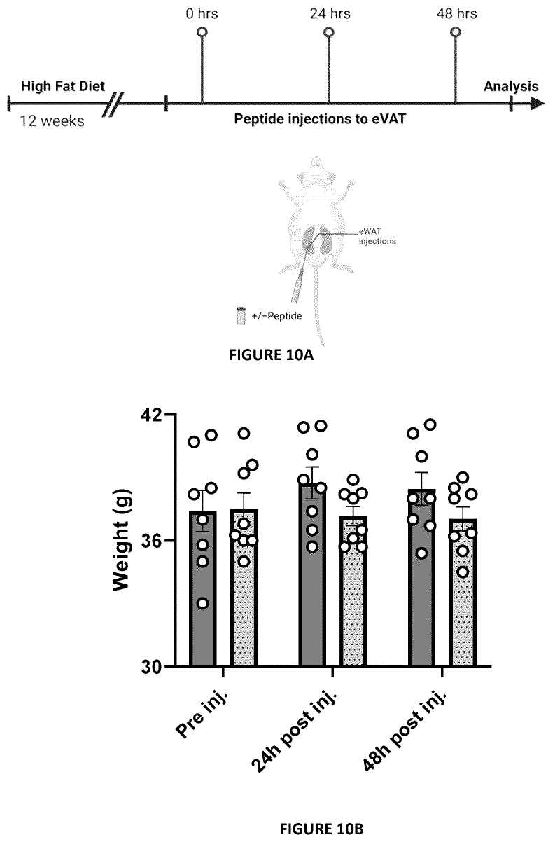

In order to evaluate the peptide's potential to mitigate the inflammatory response in a more intricate context, the inventors implemented an ex vivo analysis workflow (FIG. 9A). This approach allowed the inventors to—co-incubate the tissues with LPS and the peptide in a controlled environment, enabling examination of their combined effects on various subpopulations. For this purpose, the inventors employed epididymal adipose tissues from HFD-fed mice, chosen due to their significant immune cellular composition and proinflammatory dysfunctional properties. These tissues were subjected to a ceiling-like trans-well culture system, where LPS and the peptide were co-incubated. Tissues subjected to the combined LPS-peptide treatment exhibited a notable reduction in proinflammatory cytokine levels, with significant alterations observed in both TNFα and NOS2 levels (FIG. 9B).

Subsequently, the inventors investigated the behavior of targeted immune cells post-incubation. Following co-culture, the stromal vascular fraction was isolated, and IL6 and TNFα levels within different immune subpopulations were assessed (FIG. 9E). Remarkably, coincubation of LPS with the peptide resulted in a significant decrease in intracellular TNFα level in both general immune cells (CD45+) and adipose tissue macrophages (CD45+, CD11b, F4/80+; FIG. 9E). Additionally, to a certain extent, this coincubation exhibited a reduction in IL6 levels. They highlight the efficacy of the peptide within the complex niche of tissue, suggesting its potential effectiveness in mitigating inflammatory responses in a more physiologically relevant context.

### Example 10

### In Vivo Analysis of Adipose Tissue and the Peptide

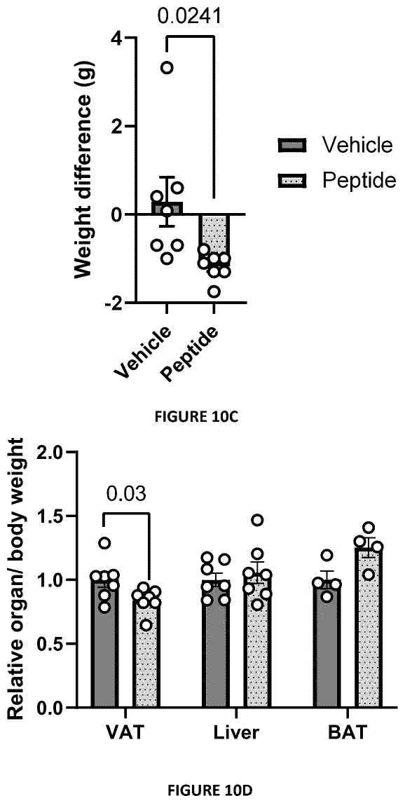

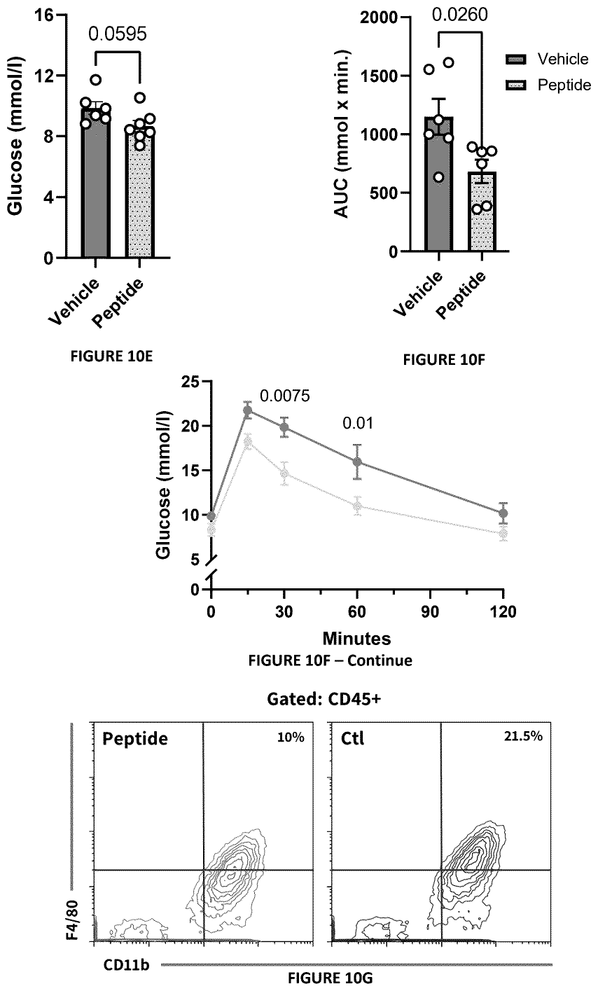

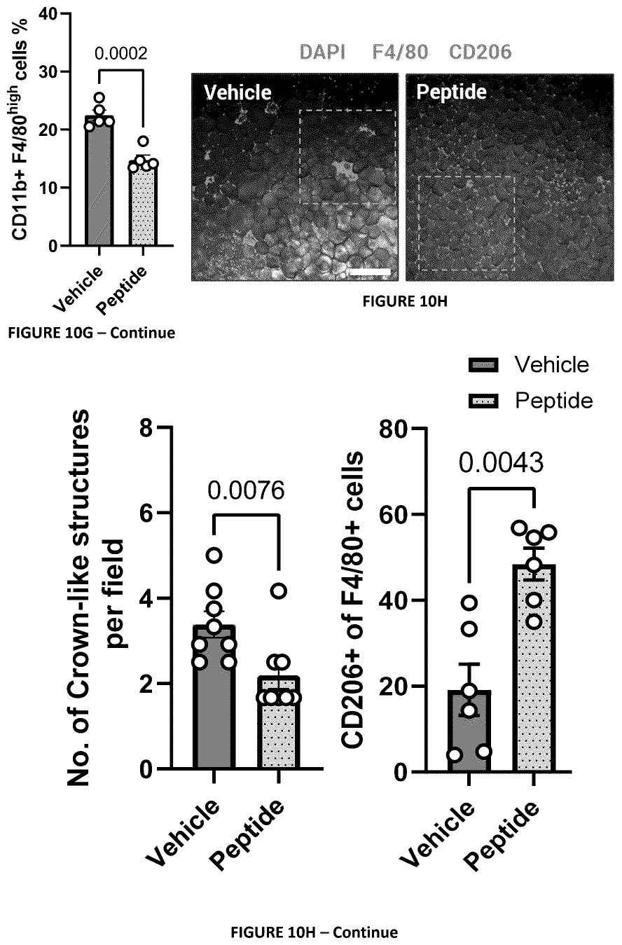

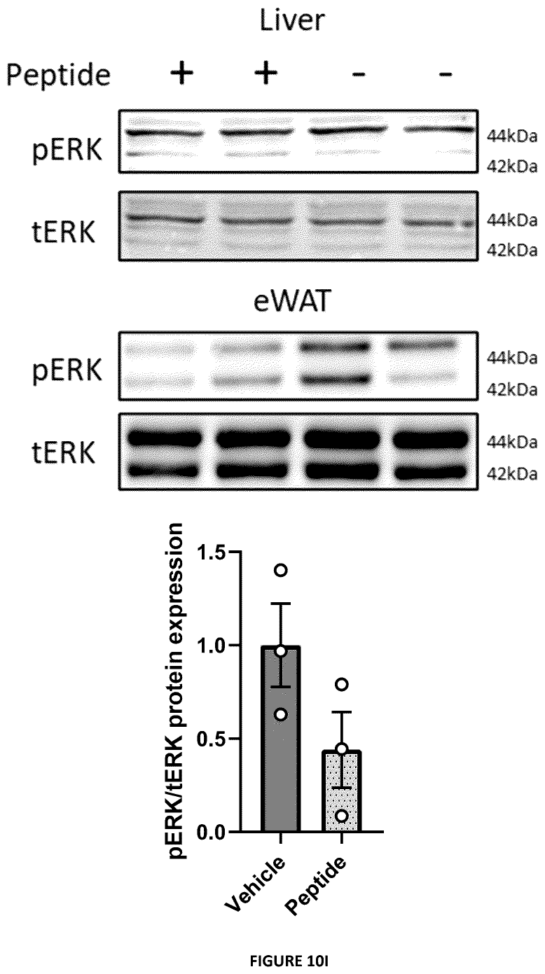

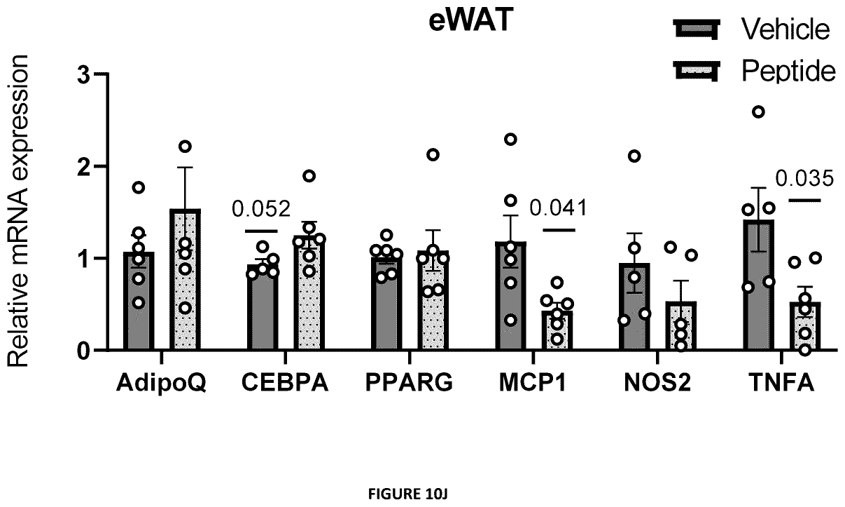

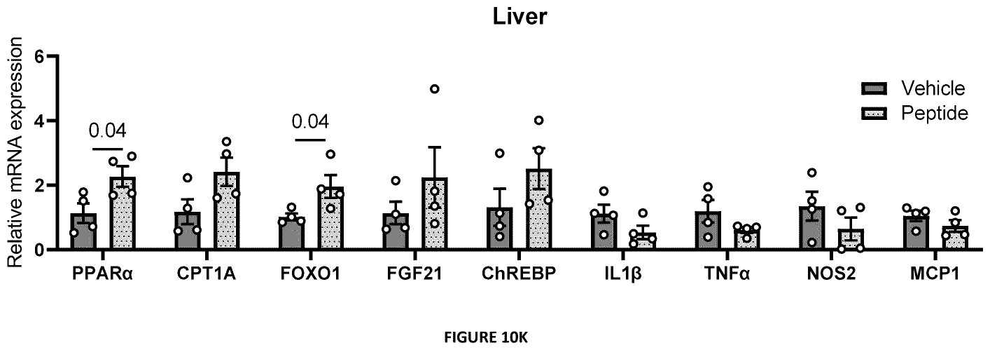

Based on the ex vivo analysis, the inventors aimed to examine the peptide's effect in vivo as a potential therapeutic agent against adipose tissue dysfunction and inflammation. To do so, the inventors generated a nutritional challenge that will lead to adipose tissue dysfunction and inflammation by feeding mice HFD for 12 weeks. These mice were then treated with the peptide and examined for any change in their metabolic status. The peptide was injected into both sides of their epididymal adipose tissue three times in 24-hour intervals at the same concentration as the in vitro assays (FIG. 10A). As shown in FIGS. 10B-10C, mice injected with the peptide lost 1.5 g on average 72 hours post the initial treatment compared to the vehicle-treated mice, which gained 1.02 g in the same period. Interestingly, the liver weights remained unchanged while the epididymal fat was reduced by 20%, suggesting a strong local effect of the peptide in the injection sites (FIG. 10D). The inventors then evaluated the glucose homeostasis and insulin sensitivity in the treated mice. Intraperitoneal glucose tolerance tests showed that the treated mice had a mild change in their responsiveness to insulin, with lower glucose levels exhibited (FIGS. 10E-10F). The inventors then analyzed the cellular immune presence in the tissue, as the peptide has been associated with the immune cells and inflammation. A decrease in both total immune cells and, more specifically, macrophages suggests that the peptide is capable of alleviating the metabolic burden by altering the immune response in the tissue (FIGS. 10G-10H). Western blot analysis revealed decreased phosphorylation of ERK in peptide-treated visceral adipose tissue, suggesting a possible inhibition of ITGA9 signaling (FIG. 10I). Interestingly, epidydimal adipose tissues demonstrated a local inflammatory relief compared to other tissues as WATs that were injected with the peptide had lower levels of both MCP1 and IL1b (FIG. 10J). Regarding the liver's metabolic function, qPCR levels of both PPARα and FOXO1, two important transcriptional regulators, were significantly upregulated, while several proinflammatory genes showed a slight decline (FIG. 10K). These results serve as the foundation for further ex vivo and in vivo work and shed light on the peptide's functionality and effectiveness in pathophysiological conditions in the adipose tissue.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for inhibiting or preventing activation of a macrophage, the method comprising contacting said macrophage with a peptide of 9 to 15 amino acids comprising the amino acid sequence: EDDMMEVPY (SEQ ID NO: 1), thereby inhibiting or preventing activation of said macrophage.

2. The method of claim 1, wherein said macrophage is characterized by expression of integrin α9β1.

3. The method of claim 1, wherein said inhibiting or preventing activation comprises reducing at least one parameter selected from the group consisting of: adhesion, migration, cell signaling, expression and/or secretion of at least one pro-inflammatory cytokine, and any combination thereof, of said macrophage.

4. The method of claim 1, wherein said macrophage is a macrophage of a subject, and optionally wherein said contacting comprises administering to said subject a therapeutically effective amount of said peptide.

5. The method of claim 1, wherein said macrophage comprises a descendant cell of a macrophage, and optionally wherein said descendant cell of a macrophage comprises a giant cell, said descendant cell of a macrophage comprises an osteoclast, or both.

6.-8. (canceled)

9. The method of claim 1, wherein said macrophage is an adipose tissue macrophage (ATM).

10. The method of claim 1, wherein said macrophage is an M1 or M1-like macrophage.

11. The method of claim 4, wherein said subject is afflicted with an inflammatory disease or disorder, and optionally wherein said inflammatory disease or disorder comprises a metabolic syndrome.

12. (canceled)

13. A method for treating or preventing an inflammatory disease or disorder, in a subject in need thereof, the method comprising administering to said subject a therapeutically effective amount of a pharmaceutical composition comprising a peptide of 9 to 15 amino acids comprising the amino acid sequence: EDDMMEVPY (SEQ ID NO: 1), thereby treating or preventing inflammatory disease or disorder, in said subject.

14. The method of claim 13, wherein said inflammatory disease or disorder comprises a metabolic syndrome, and optionally wherein said metabolic syndrome comprises any one of: obesity, pre-diabetes, diabetes, hyperglycemia, diabetic dyslipidemia, hyperlipidemia, hypertriglyceridemia, hyper-fattyacidemia, hypercholesterolemia, hyperinsulinemia, insulin-resistance, hypertension, bone resorption, and any combination thereof.

15. (canceled)

16. The method of claim 11, wherein said subject is characterized by having a macrophage being: (i) characterized by expression of integrin α9β1; (ii) an M1 or M1-like macrophage, or (iii) a combination of (i) and (ii).

17. The method of claim 13, wherein said treating or preventing comprises reducing at least one parameter selected from the group consisting of: adhesion, migration, cell signaling, expression and/or secretion of at least one pro-inflammatory cytokine, and any combination thereof, of a macrophage in said subject.

18. The method of claim 4, wherein said administering comprises: systemically administering, intravenously administering, oral administering, transdermal administering, or any combination thereof.

19. The method of claim 1, wherein said peptide is an antagonist of integrin α9β1.

20. The method of claim 1, wherein said peptide is incapable of inducing or promoting signaling via integrin α9β1.

21. The method of claim 1, wherein a level of integrin α9β1 signaling being induced by said peptide is about 0.0001%-1% the level of integrin α9β1 signaling being induced by a control ligand of integrin α9β1, and optionally wherein said control ligand of integrin α9β1 comprises an amino acid sequence set forth in SEQ ID NO: 2.

22. (canceled)

23. (canceled)

24. A method for treating or preventing a metabolic disease or disorder, in a subject in need thereof, the method comprising administering to said subject a therapeutically effective amount of a pharmaceutical composition comprising a peptide of 9 to 15 amino acids comprising the amino acid sequence: EDDMMEVPY (SEQ ID NO: 1) thereby treating or preventing a metabolic disease or disorder, in said subject.

25. The method of claim 24, wherein said metabolic disease or disorder comprises any one of: obesity, pre-diabetes, diabetes, hyperglycemia, diabetic dyslipidemia, hyperlipidemia, hypertriglyceridemia, hyper-fattyacidemia, hypercholesterolemia, hyperinsulinemia, insulin-resistance, hypertension, bone resorption, and any combination thereof.

26. The method of claim 24, wherein said treating or preventing comprises at least one parameter selected from the group consisting of: reducing weight, reducing adipose tissue volume or weight, increasing insulin sensitivity, reducing glucose level, reducing the number of inflammatory cells, reducing the number of ATMs, reducing the number of giant cells and/or osteoclasts, and any combination thereof, in said subject.

27. The method of claim 24, wherein said administering comprises injecting into a fatty tissue of said subject.
